(19) United States
(12) Patent Application Publication
(10) Pub. No.: US 2025/0331856 A1
(43) Pub. Date: Oct. 30, 2025

(54) SURGICAL INSTRUMENT

(71) Applicant: LIVSMED INC., Seongnam-si (KR)

(72) Inventor: Woo Jung CHOI, Siheung-si (KR)

(21) Appl. No.: 19/189,032

(22) Filed: Apr. 24, 2025

(30) Foreign Application Priority Data
Apr. 25, 2024 (KR) 10-2024-0055508

(51) Int. Cl. A61B 17/072; A61B 17/00

(52) U.S. Cl. CPC A61B 17/07207; A61B 2017/00367

(57) ABSTRACT

Provided is a surgical instrument that is capable of being mounted on a robot arm or operated manually for use in laparoscopic surgery or various surgeries. The surgical instrument includes an end tool that rotates in at least one direction, wherein the end tool may include a pair of jaws including a first jaw and a second jaw, which are rotatable with respect to each other, wherein the first jaw may include a first slit formed in a longitudinal direction, and the second jaw may include a second slit formed in the longitudinal direction, and an operation member including a body accommodated in the first slit and the second slit, a first flange formed on one side of the body, and a second flange formed on another side of the body, and moved in the longitudinal direction of the pair of jaws, wherein the operation member may perform an operation of closing the pair of jaws while moving from a proximal end of the pair of jaws toward a distal end and an operation of linearly moving in the longitudinal direction of the pair of jaws while the pair of jaws are closed.

### CROSS-REFERENCE TO RELATED APPLICATION

This application is based on and claims priority under 35 USC § 119 to Korean Patent Application No. 10-2024-0055508, filed on Apr. 25, 2024, in the Korean Intellectual Property Office, the disclosure of which is incorporated by reference herein in its entirety.

### BACKGROUND

### 1. Field

The present disclosure relates to a surgical instrument, and more particularly, to a surgical instrument capable of being mounted on a robot arm or operated manually for use in laparoscopic surgery or various surgeries.

### 2. Description of the Related Art

Medically, surgery refers to the treatment of diseases by cutting, slitting, or manipulating the skin, mucous membranes, or other tissues using medical devices In particular, open surgery, which cuts and opens the skin of a surgical site and cures, shapes, or removes an organ therein, may cause bleeding, side effects, patient pain, scars, or the like. Accordingly, recently, surgery performed by inserting only a medical device, for example, laparoscopic surgical instrument, microsurgical microscope, and the like by forming a predetermined hole in the skin or surgery using a robot has been spotlighted as an alternative.

Here, a surgical robot refers to a robot that has the function of replacing surgical actions performed by a surgeon. Compared to humans, the surgical robot has the advantage of being able to operate with greater accuracy and precision, as well as being able to operate remotely.

Surgical robots that are currently being developed worldwide may include a bone surgical robot, a laparoscopic surgical robot, a stereotactic surgical robot, and the like. Here, the laparoscopic surgical robot is a robot that performs minimum invasive surgery using a laparoscope and small surgical instruments.

The laparoscopic surgery is a surgical method in which one or more small holes are drilled in the abdomen of a patient and the inside of the abdominal cavity is observed through these holes, and is widely used in general surgery and the like. Today's laparoscopes are mounted with computer chips and have been developed to the extent that magnified images, which are clearer than images seen with the naked eye, can be obtained and when used with specially-designed laparoscopic surgical tools while looking at a monitor screen, any type of surgery is possible.

Moreover, laparoscopic surgery offers the same range of surgical procedures as open surgery, but with several advantages including fewer complications, the ability to initiate treatment shortly after the procedure, and the capability to maintain the patient's stamina and immune functions. As a result, laparoscopic surgery is becoming increasingly recognized as the standard surgery for treating colorectal cancer or the like in places such as the United States and Europe.

Meanwhile, a surgical robot is generally composed of a master robot and a slave robot. When a surgical operator manipulates a control lever (e.g., a handle) equipped on the master robot, a surgical tool coupled to or held by a robot arm on the slave robot may be manipulated to perform surgery.

The background art described above is technical information retained by the present inventors in order to derive the present disclosure or obtained by the present inventors in the process of deriving the present disclosure, and thus is not necessarily known art disclosed to the general public before the filing of the present application.

### SUMMARY

The present disclosure is directed to providing a surgical instrument capable of efficiently moving an operation member along a jaw by applying a minimum load to the operation member.

However, the object is illustrative, and the object to be solved by the present disclosure is not limited thereto.

Additional aspects will be set forth in part in the description which follows and, in part, will be apparent from the description, or may be learned by practice of the presented embodiments of the disclosure.

According to an aspect of the present invention, there is provided a surgical instrument including an end tool that rotates in at least one direction, wherein the end tool may include a pair of jaws including a first jaw and a second jaw, which are rotatable with respect to each other, wherein the first jaw may include a first slit formed in a longitudinal direction, and the second jaw may include a second slit formed in the longitudinal direction, and an operation member including a body accommodated in the first slit and the second slit, a first flange formed on one side of the body, and a second flange formed on another side of the body, and moved in the longitudinal direction of the jaw, wherein the operation member may perform an operation of closing the jaws while moving from a proximal end of the jaw toward a distal end and an operation of linearly moving in the longitudinal direction of the jaw while the jaws are closed.

Further, the second jaw may include a jaw-inclined surface formed to be inclined upward in a direction toward the distal end of the second jaw.

Further, the jaw-inclined surface may be formed such that an inclination angle changes in the longitudinal direction.

Further, the jaw-inclined surface may be formed such that an inclination angle at a proximal end is greater than an inclination angle at a distal end.

Further, a distance between a surface of the second jaw, with which the first flange comes into contact, and a surface of the first jaw, with which the second flange comes into contact, may be formed to have a value greater than or equal to that of a vertical distance between the first jaw and the second jaw, and the operation member may be subjected to a rotational force while moving in the longitudinal direction of the jaw.

Further, when performing the operation of closing the jaws, the operation member may be subjected to a rotational force in a direction that causes the second flange to move forward further toward the distal end of the jaw than the first flange.

Further, when the operation member performs the operation of closing the jaws, a distal end of the first flange may come into contact with the jaw-inclined surface, and a distal end of the second flange may come into contact with the first jaw.

Further, when the operation member is moved in a direction toward the distal end of the jaw while the jaws are closed, the second flange may be subjected to a rotational force in a direction that causes the second flange to move forward further toward the distal end than the first flange.

Further, when the operation member is moved in the direction toward the distal end of the jaw while the jaws are closed, a proximal end of the first flange may come into contact with the second jaw and a distal end of the second flange may come into contact with the first jaw.

Further, when a horizontal distance from a rotation axis of the operation member to a proximal end of the first flange is n [mm], and a horizontal distance from the rotation axis of the operation member to a distal end of the second flange is o [mm], a relationship of n+o>10 mm may be satisfied.

Further, the operation member may be formed such that the second flange extends further in a direction toward the distal end than the first flange.

Further, the first flange may have a length of 2 to 10 mm.

Further, when a length of the first flange is m [mm] and a length by which the second flange extends further than the first flange is l [mm], a relationship of m/l<10 may be satisfied.

Further, the second flange may extend further than the first flange to a length whose value is greater than a value that causes a sum of rotational moments applied to the operation member to be zero.

In addition, when the inclination angle of the jaw-inclined surface is w [degree] and the length of the second flange extending beyond the first flange is l [mm], w/l<5 [degree/mm] may be satisfied.

Further, a width of the body may be formed to be less than a width of each of the first slit and the second slit.

Further, the operation member may further include a third flange formed parallel to the second flange and formed at a proximal end of the operation member.

Further, at least one of the first flange and the second flange may include a bent part formed to be curved at a portion in contact with the jaw.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a perspective view illustrating a surgical instrument according to an embodiment of the present disclosure;

FIG. 2 is a side view of the surgical instrument of FIG. 1;

FIG. 3 is a perspective view illustrating an end tool of the surgical instrument of FIG. 1;

FIGS. 4 and 5 are exploded perspective views of the end tool of the surgical instrument of FIG. 3;

FIG. 6 is an exploded perspective view illustrating one embodiment of the staple drive assembly of the surgical instrument of FIG. 1;

FIG. 7 is a plan view illustrating a first jaw of the end tool of the surgical instrument of FIG. 3;

FIG. 8 is a plan view illustrating a second jaw of the end tool of the surgical instrument of FIG. 3;

FIGS. 9 and 10 are exploded perspective views illustrating one modified example of the staple drive assembly of the surgical instrument of FIG. 1;

FIGS. 11 and 12 are side views of the staple drive assembly of FIGS. 9 and 10;

FIGS. 13 and 14 are views for describing operations of the staple drive assembly of FIGS. 9 to 12;

FIG. 15 is a perspective view illustrating the first jaw and a cartridge of the surgical instrument of FIG. 1;

FIG. 16 is an exploded perspective view of the cartridge of FIG. 15;

FIG. 17 is a perspective view of the cartridge of FIG. 15;

FIG. 18 is a side view of the cartridge of FIG. 15;

FIG. 19 is a perspective cross-sectional view for describing an internal structure of the cartridge of FIG. 15;

FIG. 20 is a side cross-sectional view for describing the internal structure of the cartridge of FIG. 15;

FIGS. 21A to 21C are views for describing a staple motion of the end tool of FIG. 3;

FIG. 22 is a view schematically illustrating the surgical instrument according to an embodiment of the present disclosure;

FIG. 23 is a view for describing operations of an operation member of the surgical instrument of FIG. 22;

FIGS. 24 and 25 are perspective views illustrating the jaws of FIG. 22;

FIG. 26 is a perspective view illustrating the operation member of FIG. 23;

FIG. 27 is a view with a wedge omitted from FIG. 26;

FIG. 28 is a side view of FIG. 27;

FIG. 29 is a perspective view illustrating one modified example of the operation member of FIG. 27;

FIG. 30 is a side view of FIG. 29.

FIG. 31 is a view illustrating a state before the operation member of FIG. 23 performs an operation (clamping operation) of closing the jaws;

FIG. 32 is an enlarged view of portion Z1 of FIG. 31;

FIG. 33 is a view illustrating a state in which the operation member of FIG. 23 performs the operation (clamping operation) of closing the jaws;

FIG. 34 is an enlarged view of portion Z2 of FIG. 33;

FIG. 35 is a view illustrating a state after the operation member of FIG. 23 performs the operation (clamping operation) of closing the jaws;

FIG. 36 is an enlarged view of portion Z3 of FIG. 35;

FIG. 37 is a view for describing operations of the operation member when the operation member performs the operation (clamping operation) of closing the jaws;

FIG. 38 is a view for describing operations of the operation member when the operation member performs an operation of moving along the jaw; and

FIG. 39 is a side view schematically illustrating the operation member of FIGS. 37 and 38.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects. As used herein, the term “and/or” includes any and all combinations of one or more of the associated listed items. Expressions such as “at least one of,” when preceding a list of elements, modify the entire list of elements and do not modify the individual elements of the list.

While the present disclosure is susceptible to various modifications and alternative forms, specific embodiments thereof are shown by way of example in the drawings and will herein be described in detail. Advantages and features of the present disclosure and methods for accomplishing the same will be more clearly understood from embodiments described below with reference to the drawings. However, the present disclosure is not limited to the embodiments disclosed below but may be implemented in various forms.

Hereinafter, the embodiments of the present disclosure will be described in detail with reference to the accompanying drawings, but when describing with reference to the drawings, equal or corresponding components will be referred to as the same reference numerals, and redundant descriptions thereof will be omitted.

In the following embodiments, the terms “first,” “second,” and the like have been used to distinguish one component from another, rather than limitative in all aspects.

In the following embodiments, singular forms are intended to include plural forms as well, unless the context clearly indicates otherwise.

In the following embodiments, terms such as “include” or “have” means that the features or components described in the specification are present, and the possibility that one or more other features or components will be added is not excluded in advance.

Sizes of components in the drawings may be exaggerated or reduced for convenience of description. For example, the size and thickness of each component shown in the drawings are arbitrarily represented for convenience of description, and thus, the present disclosure is not necessarily limited thereto.

In the following embodiments, an X-axis, a Y-axis, and a Z-axis are not limited to three axes of the rectangular coordinate system, and may be interpreted in a broader sense. For example, the X-axis, the Y-axis, and the Z-axis may be perpendicular to one another, or may represent different directions that are not perpendicular to one another.

In cases where certain embodiments may be implemented otherwise, a specific process sequence may be performed differently from the described sequence. For example, two consecutively described processes may be performed substantially at the same time or performed in an order opposite to the described order.

Hereinafter, based on the above principle, a surgical instrument according to the present disclosure will be described in detail with reference to the drawings.

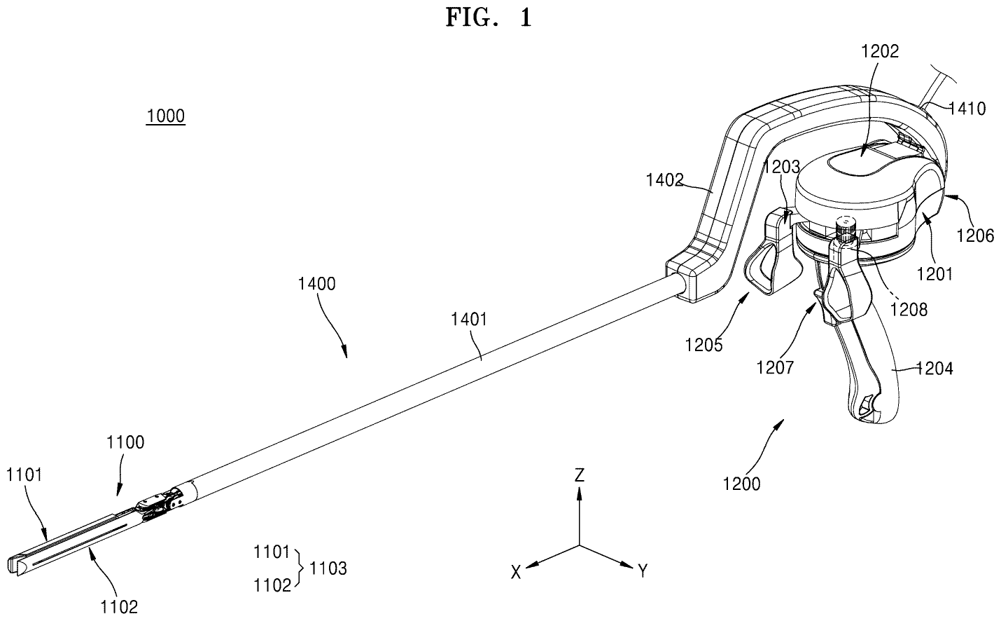

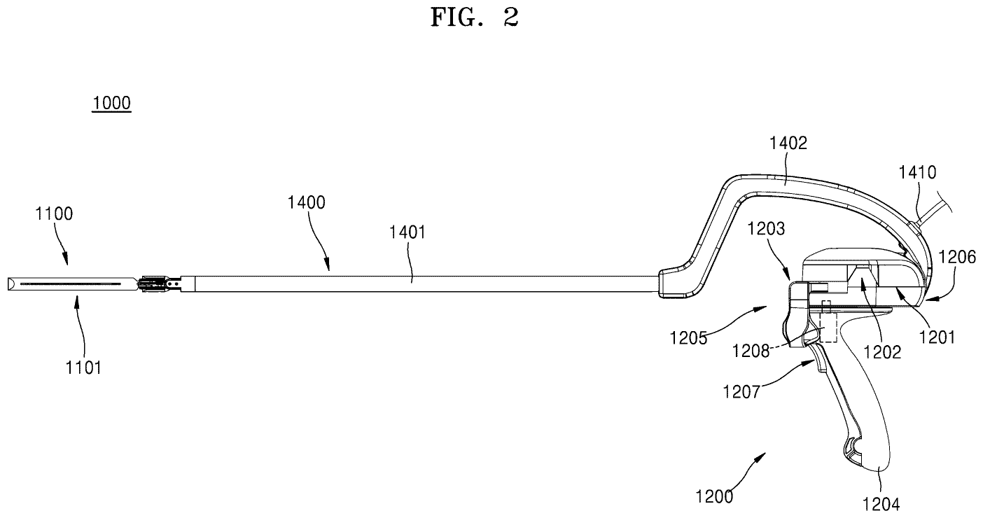

FIG. 1 is a perspective view illustrating a surgical instrument according to an embodiment of the present disclosure, and FIG. 2 is a side view of the surgical instrument of FIG. 1.

First, referring to FIGS. 1 and 2, a surgical instrument 1000 according to the present embodiment includes an end tool 1100, a manipulation part 1200, a power transmission part (not shown), and a connection part 1400.

Here, the connection part 1400 is formed in the shape of a hollow shaft, and one or more wires and electric wires may be accommodated therein. The manipulation part 1200 is coupled to one end portion of the connection part 1400, the end tool 1100 is coupled to another end portion thereof, and the connection part 1400 may serve to connect the manipulation part 1200 to the end tool 1100. The connection part 1400 includes a straight part 1401 and a bent part 1402, the straight part 1401 is formed on a side of the connection part 1400 coupled to the end tool 1100, and the bent part 1402 is formed on a side of the connection part 1400 to which the manipulation part 1200 is coupled. As such, since the end portion of the connection part 1400 at the side of the manipulation part 1200 is formed to be bent, a pitch manipulation part 1201, a yaw manipulation part 1202, and an actuation manipulation part 1203 may be formed along an extension line of the end tool 1100 or adjacent to the extension line. In other words, it may be described that the pitch manipulation part 1201 and the yaw manipulation part 1202 are at least partially accommodated in a concave portion formed by the bent part 1402. Due to the above-described shape of the bent part 1402, the shapes and motions of the manipulation part 1200 and the end tool 1100 may be further intuitively matched with each other.

Meanwhile, a plane on which the bent part 1402 is formed may be substantially the same as a pitch plane, that is, an XZ plane of FIG. 1. As such, as the bent part 1402 is formed on substantially the same plane as the XZ plane, interference with the manipulation part may be reduced. Of course, for intuitive motions of the end tool and the manipulation part, any form other than the X Z plane may be possible.

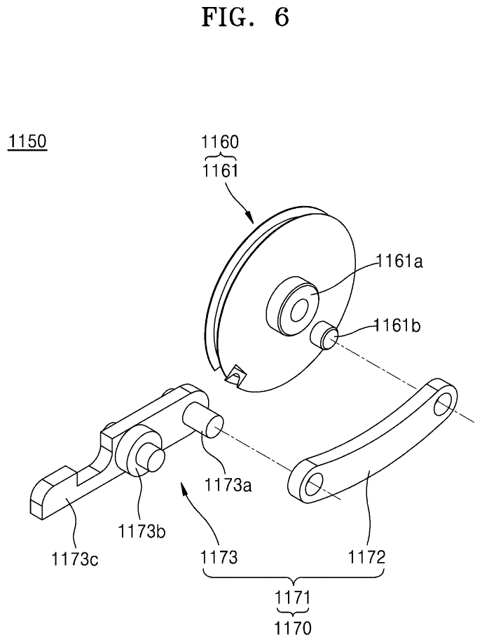

Meanwhile, a connector 1410 may be formed on the bent part 1402. The connector 1410 may be connected to an external power source (not shown), and the connector 1410 may also be connected to the end tool 1100 via an electric wire, and may transmit, to the end tool 1100, electric energy supplied from the external power source (not shown). In addition, the electric energy transmitted to the end tool 1100 as described above may produce a driving force for rotating a staple pulley (see 1161 in FIG. 6) to be described below in a clockwise or counterclockwise direction. In addition, the electric energy may be supplied to drive a driving part 1208. Of course, a built-in battery may be used.

The manipulation part 1200 is formed at the one end portion of the connection part 1400 and provided as an interface to be directly controlled by a medical doctor, and may have, for example, a tongs shape, a stick shape, a lever shape, or the like, and when the medical doctor controls the manipulation part 1200, the end tool 1100, which is connected to the corresponding interface and inserted into the body of a surgical patient, performs a certain motion, thereby performing surgery. Here, the manipulation part 1200 is illustrated in FIGS. 1 and 2 and the like as being formed in a handle shape that is rotatable while the finger is inserted therein, but the concept of the present disclosure is not limited thereto, and various types of manipulation parts that can be connected to the end tool 1100 and manipulate the end tool 1100 may be possible.

The end tool 1100 is formed on another end portion of the connection part 1400, and performs necessary motions for surgery by being inserted into a surgical site. As an example of the end tool 1100 described above, a pair of jaws 1103 for performing a grip motion may be used as shown in FIG. 1. However, the concept of the present disclosure is not limited thereto, and various devices for performing surgery may be used as the end tool 1100. For example, a configuration such as a cantilever cautery may also be used as the end tool. The end tool 1100 is connected to the manipulation part 1200 by the power transmission part (not shown), and receives a driving force of the manipulation part 1200 through the power transmission part (not shown) to perform a motion necessary for surgery, such as gripping, cutting, suturing, or the like.

Here, the end tool 1100 of the surgical instrument 1000 according to an embodiment of the present disclosure is formed to be rotatable in at least one direction, for example, the end tool 1100 may perform a pitch motion around a Y-axis of FIG. 1 and simultaneously perform a yaw motion and an actuation motion around a Z-axis of FIG. 1.

Here, each of the pitch, yaw, and actuation motions used in the present disclosure are defined as follows.

First, the pitch motion means a motion of the end tool 1100 rotating in a vertical direction with respect to an extension direction of the connection part 1400 (an X-axis direction of FIG. 1), that is, a motion rotating around the Y-axis of FIG. 1. In other words, the pitch motion means a motion of the end tool 1100, which is formed to extend from the connection part 1400 in the extension direction of the connection part 1400 (the X-axis direction of FIG. 1), rotating vertically around the Y-axis with respect to the connection part 1400.

Next, the yaw motion means a motion of the end tool 1100 rotating in left and right directions, that is, a motion rotating around the Z-axis of FIG. 1, with respect to the extension direction of the connection part 1400 (the X-axis direction of FIG. 1). In other words, the yaw motion means a motion of the end tool 1100, which is formed to extend from the connection part 1400 in the extension direction of the connection part 1400 (the X-axis direction of FIG. 1), rotating horizontally around the Z-axis with respect to the connection part 1400. That is, the yaw motion means a motion of the two jaws 1103, which are formed on the end tool 1100, rotating around the Z-axis in the same direction.

Meanwhile, the actuation motion may mean a motion of the end tool 1100 rotating around the same axis of rotation as that of the yaw motion, while the two jaws 1103 rotate in opposite directions so as to be closed or opened. That is, the actuation motion means rotating motions of the two jaws 1103, which are formed on the end tool 1100, in opposite directions around the Z-axis.

The power transmission part (not shown) may connect the manipulation part 1200 to the end tool 1100, transmit the driving force of the manipulation part 1200 to the end tool 1100, and include a plurality of wires, pulleys, links, sections, gears, or the like.

The end tool 1100, the manipulation part 1200, and the like of the surgical instrument 1000 of FIG. 1 will be described in detail below.

### Intuitive Driving

Hereinafter, intuitive driving of the surgical instrument 1000 of the present disclosure will be described.

First, a user may perform a pitch motion by rotating a first handle 1204 around the Y-axis while holding the first handle 1204 with a palm thereof, and perform a yaw motion by rotating the first handle 1204 around the Z-axis. In addition, the user may perform an actuation motion by manipulating the actuation manipulation part 1203 in a state in which the thumb and the index finger are inserted into a hand ring-shaped extension part formed at one end portion of the actuation manipulation part 1203.

Here, in the surgical instrument 1000 according to an embodiment of the present disclosure, when the manipulation part 1200 is rotated in one direction with respect to the connection part 1400, the end tool 1100 is rotated in a direction that is intuitively the same as a manipulation direction of the manipulation part 1200. In other words, when the first handle 1204 of the manipulation part 1200 is rotated in one direction, the end tool 1100 is also rotated in a direction intuitively the same as the one direction, so that a pitch motion or a yaw motion is performed. Here, the phrase “intuitively the same direction” may be further explained as meaning that a direction of movement of the user's finger gripping the manipulation part 1200 and a direction of movement of a distal end of the end tool 1100 form substantially the same direction. Of course, “the same direction” as used herein may not be a perfectly matching direction on a three-dimensional coordinate, and may be understood to be equivalent to the extent that, for example, when the user's finger moves to the left, the distal end of the end tool 1100 is moved to the left, and when the user's finger moves down, the end portion of the end tool 1100 is moved down.

In addition, to this end, in the surgical instrument 1000 according to the present embodiment, the manipulation part 1200 and the end tool 1100 are formed in the same direction with respect to a plane perpendicular to the extension axis (X-axis) of the connection part 1400. That is, when viewed based on a Y Z plane of FIG. 1, the manipulation part 1200 is formed to extend in a positive (+) X-axis direction, and the end tool 1100 is also formed to extend in the positive (+) X-axis direction. In other words, it may be said that a formation direction of the end tool 1100 on one end portion of the connection part 1400 is the same as a formation direction of the manipulation part 1200 on another end portion of the connection part 1400 on the basis of the Y Z plane. Further, in other words, it may be said that the manipulation part 1200 may be formed in a direction away from the body of a user holding the manipulation part 1200, that is, in a direction in which the end tool 1100 is formed. That is, the first handle 1204 or the like, which the user grips and moves to perform the actuation motion, the yaw motion, the pitch motion, and the like, is formed such that the portion that moves to perform each motion extends in the positive (+) X-axis direction beyond the center of rotation of each joint for that motion. In this manner, the manipulation part 1200 may be configured in the same manner as the end tool 1100 in which each moving portion is formed to extend in the positive (+) X-axis direction from the rotation center of a corresponding joint for the motion, and the manipulation direction of the user may be identical to an operation direction of the end tool from the viewpoint of the rotation directions and the left and right directions. As a result, intuitively the same manipulation may be achieved.

In detail, in the case of the conventional surgical instrument, a direction in which a user manipulates the manipulation part is different from a direction in which the end tool is actually operated, that is, intuitively different from the direction in which the end tool is actually operated, and thus, a surgical operator may not easily intuitively manipulate the surgical instrument and may spend a long time to learn a skill of operating the end tool in desired directions, and in some cases, malfunctions may occur, which may cause damage to patients.

In order to address such problems, the surgical instrument 1000 according to an embodiment of the present disclosure is configured such that the manipulation direction of the manipulation part 1200 and the operation direction of the end tool 1100 are intuitively identical to each other. To this end, the manipulation part 1200 is configured similar to the end tool 1100, that is, in the manipulation part 1200, portions that are actually moved for actuation, yaw, and pitch motions extend respectively from rotation centers of corresponding joints in the positive (+) X-axis direction.

Hereinafter, the end tool 1100, the manipulation part 1200, and the like of the surgical instrument 1000 of FIG. 1 will be described in more detail.

### End Tool

Hereinafter, the end tool 1100 of the surgical instrument 1000 will be described in more detail.

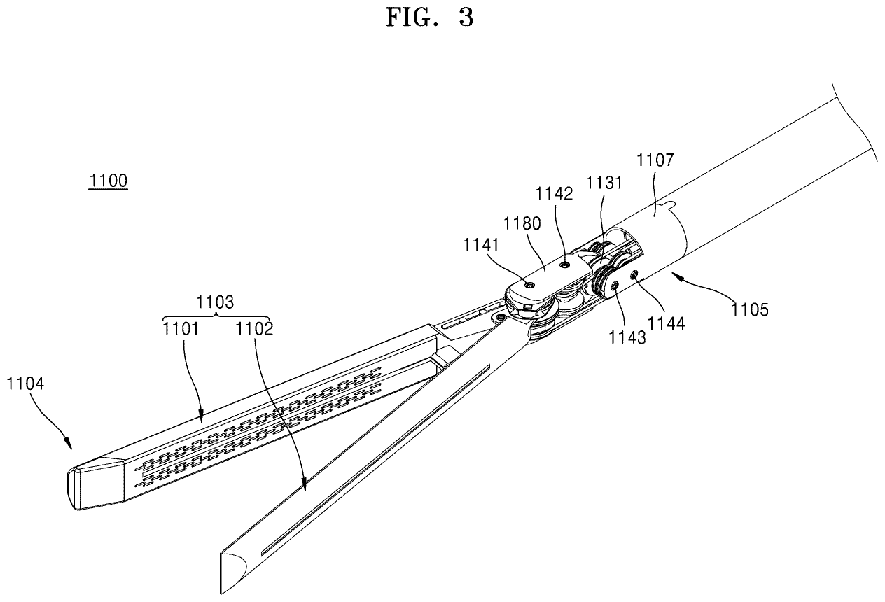

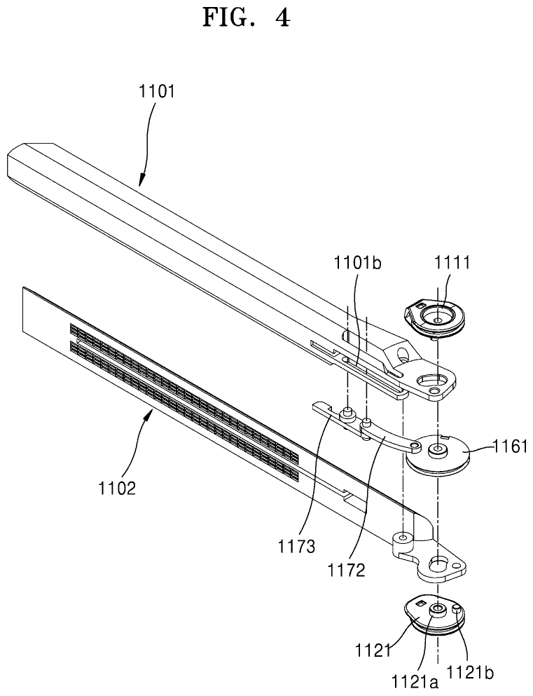

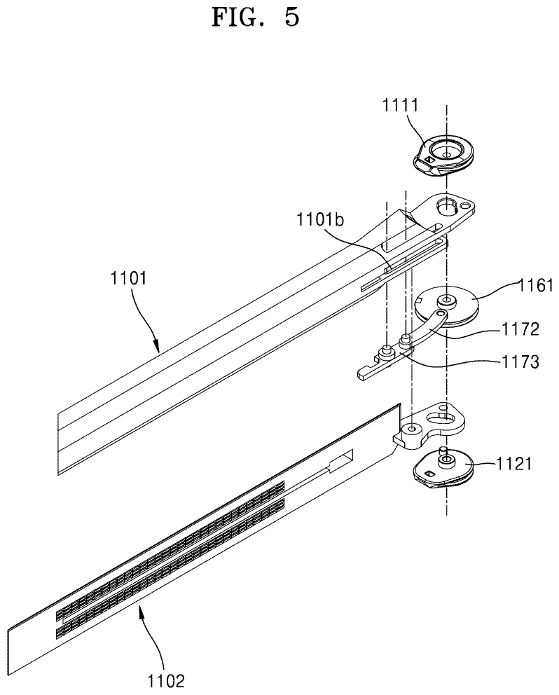

FIG. 3 is a perspective view illustrating the end tool of the surgical instrument of FIG. 1, and FIGS. 4 and 5 are exploded perspective views of the end tool of the surgical instrument of FIG. 3.

The end tool 1100 of the surgical instrument according to an embodiment of the present disclosure includes a pair of jaws for performing a grip motion, that is, a first jaw 1101 and a second jaw 1102. Here, each of the first jaw 1101 and the second jaw 1102, or a component encompassing the first jaw 1101 and the second jaw 1102 may be referred to as the jaw 1103.

In addition, the end tool 1100 may include a plurality of pulleys including a pulley 1111 associated with a rotational motion of the first jaw 1101. In addition, the end tool 1100 may include a plurality of pulleys, including a pulley 1121 associated with rotational movement of the second jaw 1102.

Here, the pulleys facing each other are illustrated in the drawings as being formed parallel to each other, but the concept of the present disclosure is not limited thereto, and each of the pulleys may be variously formed with a position and a size suitable for the configuration of the end tool.

Further, the end tool 1100 of the present embodiment may include an end tool hub 1180 and a pitch hub 1107.

A rotation shaft 1141 and a rotation shaft 1142 may be inserted through the end tool hub 1180, and the end tool hub 1180 may internally accommodate at least some of one or more pulleys that are axially coupled to the rotation shaft 1141. In addition, the end tool hub 1180 may internally accommodate at least some of one or more pulleys that are axially coupled to the rotation shaft 1142.

In addition, at least some of a staple pulley assembly (see 1160 of FIG. 6) and a staple link assembly (see 1170 of FIG. 6) to be described below may be formed at one side of the end tool hub 1180, e.g., in a space adjacent to the center of the end tool hub 1180.

Meanwhile, a pulley 1131 serving as an end tool pitch pulley may be formed at one end portion of the end tool hub 1180. Alternatively, the pulley 1131 may be integrally formed with the end tool hub 1180 as one body. That is, a disk-shaped pulley is formed at one end portion of the end tool hub 1180, and a groove around which a wire may be wound may be formed on an outer circumferential surface of the pulley. Alternatively, the pulley 1131 may be formed as a separate member from the end tool hub 1180 to be coupled to the end tool hub 1180.

A rotation shaft 1143 and a rotation shaft 1144 are inserted through the pitch hub 1107, and the pitch hub 1107 may be axially coupled to the end tool hub 1180 (and the pulley 1131) by the rotation shaft 1143. Thus, the end tool hub 1180 and the pulley 1131 may be formed to be rotatable around the rotation shaft 1143 with respect to the pitch hub 1107.

In addition, the pitch hub 1107 may internally accommodate at least some of one or more pulleys that are axially coupled to the rotation shaft 1143. In addition, the pitch hub 1107 may internally accommodate at least some of one or more pulleys that are axially coupled to the rotation shaft 1144.

In addition, the end tool 1100 of the present embodiment may include the rotation shaft 1141, the rotation shaft 1142, the rotation shaft 1143, and the rotation shaft 1144. As described above, the rotation shaft 1141 and the rotation shaft 1142 may be inserted through the end tool hub 1180, and the rotation shaft 1143 and the rotation shaft 1144 may be inserted through the pitch hub 1107.

The rotation shaft 1141, the rotation shaft 1142, the rotation shaft 1143, and the rotation shaft 1144 may be arranged sequentially from a distal end 1104 of the end tool 1100 toward a proximal end 1105. Accordingly, starting from the distal end 1104, the rotation shaft 1141 may be referred to as a first pin, the rotation shaft 1142 may be referred to as a second pin, the rotation shaft 1143 may be referred to as a third pin, and the rotation shaft 1144 may be referred to as a fourth pin.

Here, the rotation shaft 1141 may function as an end tool jaw pulley rotation shaft, the rotation shaft 1142 may function as an end tool jaw auxiliary pulley rotation shaft, the rotation shaft 1143 may function as an end tool pitch rotation shaft, and the rotation shaft 1144 may function as an end tool pitch auxiliary rotation shaft of the end tool 1100.

One or more pulleys may be inserted into each of the rotation shafts 1141 1142, 1143, and 1144.

Meanwhile, a rotation shaft may be further formed on one side of the rotation shaft 1141, specifically, one side of the rotation shaft 1141 at the distal end 1104 side.

The pulley 1111 functions as an end tool first jaw pulley, and the pulley 1121 functions as an end tool second jaw pulley. The pulley 1111 may also be referred to as a first jaw pulley, and the pulley 1121 may also be referred to as a second jaw pulley, and these two components may also be referred to collectively as an end tool jaw pulley or simply a jaw pulley.

The pulley 1111 and the pulley 1121, which are end tool jaw pulleys, are formed to face each other, and are formed to be rotatable independently of each other around the rotation shaft 1141, which is an end tool jaw pulley rotation shaft. In this case, the pulley 1111 and the pulley 1121 are formed to be spaced apart from each other by a certain extent, and a staple assembly accommodation part may be formed therebetween. In addition, at least some of the staple pulley assembly 1160 and the staple link assembly 1170, which will be described below, may be disposed inside the staple assembly accommodation part.

Here, in the drawings, it is illustrated that the pulley 1111 and the pulley 1121 are formed to rotate around one rotation shaft 1141, but it is of course possible that each end tool jaw pulley may be formed to be rotatable around a separate shaft. Here, the first jaw 1101 is fixedly coupled to the pulley 1111 and rotated together with the pulley 1111, and the second jaw 1102 is fixedly coupled to the pulley 1121 and rotated together with the pulley 1121. Y aw and actuation motions of the end tool 1100 are performed in response to the rotation of the pulley 1111 and the pulley 1121. That is, when the pulley 1111 and the pulley 1121 are rotated in the same direction around the rotation shaft 1141, the yaw motion is performed, and when the pulley 1111 and the pulley 1121 are rotated in opposite directions around the rotation shaft 1141, the actuation motion is performed.

Here, the first jaw 1101 and the pulley 1111 may be formed as separate members and coupled to each other, or the first jaw 1101 and the pulley 1111 may be integrally formed as one body. Similarly, the second jaw 1102 and the pulley 1121 may be formed as separate members and coupled to each other, or the second jaw 1102 and the pulley 1121 may be integrally formed as one body.

In addition, one or more auxiliary pulleys may be disposed adjacent to the pulley 1111 and the pulley 1121.

These pulleys may be formed such that one or more wires are wound therearound, the pulleys may be rotated by the wires, and the wires may move along the pulleys, thereby transmitting a driving force to the end tool 1100.

### Components Related to Staple Pulley

Hereinafter, a staple drive assembly the end tool 1100 of the surgical instrument of FIG. 1 will be described in more detail.

FIG. 6 is an exploded perspective view illustrating one embodiment of the staple drive assembly of the surgical instrument of FIG. 1.

The end tool 1100 of the present embodiment may include one or more pulleys including the staple pulley 1161 associated with linear motion/rotational motion of the respective pulleys and links for stapling and cutting.

The staple pulley 1161 is formed to face each of the pulley 1111 and the pulley 1121, which are end tool jaw pulleys, and the staple pulley 1161, the pulley 1111, and the pulley 1121 are formed to be rotatable independently of each other around the rotation shaft 1141, which is an end tool jaw pulley rotation shaft. Here, the staple pulley 1161 is illustrated as being disposed between the pulley 1111 and the pulley 1121, but the concept of the present disclosure is not limited thereto, and the staple pulley 1161 may be disposed at various positions adjacent to the pulley 1111 or the pulley 1121.

Here, in the present disclosure, the staple pulley 1161, the pulley 1111, and the pulley 1121 are formed to rotate around substantially the same shaft. As the staple pulley 1161, the pulley 1111, and the pulley 1121 are formed to rotate around the same shaft as described above, it is possible to perform a pitch motion/yaw motion/actuation motion as well as stapling and cutting motions.

Although the staple pulley 1161, the pulley 1111, and the pulley 1121 are illustrated in the drawing as being formed to rotate around one rotation shaft 1141, it is of course possible that each jaw pulley may be formed to be rotatable around a separate shaft that is concentric therewith.

In other words, it may also be described as a structure in which the pulley 1111, which is a first jaw pulley, the staple pulley 1161, and the pulley 1121 that is a second jaw pulley are sequentially stacked along the rotation shaft 1141. Alternatively, it may be also described as a structure in which the staple pulley 1161 is disposed between the pulley 1111 and the pulley 1121 facing each other. Here, the pulley 1111, which is a first jaw pulley, the staple pulley 1161, and the pulley 1121 that is a second jaw pulley may be formed to be rotatable independently of each other.

One or more staple auxiliary pulleys (not shown) may further be provided on one side of the staple pulley 1161.

In addition, one or more pulleys (not shown) may function as staple pitch main pulleys, and another one or more pulleys may function as staple pitch sub-pulleys.

As one or more wires are pulled and released by the driving part 1208 of FIG. 1, the staple pulley 1161 coupled thereto may be rotated in one direction, and stapling may be performed accordingly.

In addition, in an optional embodiment, one or more staple auxiliary pulleys (not shown) are disposed to increase a radius of rotation of the staple pulley 1161, thereby increasing a yaw motion range in which normal stapling and cutting operations may be performed.

### Staple Drive Assembly

Hereinafter, a staple drive assembly 1150 will be described in more detail.

Referring to FIG. 6 and the like, the staple drive assembly 1150 may include the staple pulley assembly 1160 and the staple link assembly 1170. Here, the staple drive assembly 1150 is connected to a moving member 1550 of a cartridge 1500 to be described below, and converts a rotational motion of the staple pulley 1161 into a linear motion of the moving member 1550. In other embodiments of the present disclosure, which will be described below, the staple drive assembly may be understood as a concept including the staple pulley assembly and the staple link assembly. A driving force is transmitted to the staple drive assembly 1150 through the driving part 1208, and the moving member 1550 and the operation member 1540 connected thereto are moved due to the movement of the staple drive assembly 1150.

The staple pulley assembly 1160 may include one or more staple pulleys 1161. The staple pulley assembly 1160 may be formed between the pulley 1111 and the pulley 1121 to be adjacent to the pulley 1111 and the pulley 1121. In the present embodiment, it is assumed that the staple pulley assembly 1160 includes one staple pulley 1161.

A shaft pass-through part 1161a may be formed in the staple pulley 1161. The shaft pass-through part 1161a may be formed in the form of a hole, and the rotation shaft 1141, which is an end tool jaw pulley rotation shaft, may be inserted through the shaft pass-through part 1161a. In addition, a link coupling part 1161b may be formed on the staple pulley 1161. The staple link assembly 1170 to be described below may be coupled to the link coupling part 1161b. This will be described in more detail later.

Meanwhile, the end tool 1100 of the present embodiment may further include the staple link assembly 1170 connected to the staple pulley assembly 1160. The staple link assembly 1170 may include one or more link members 1171. The staple link assembly 1170 may serve to connect the staple pulley assembly 1160 to the moving member 1550 of the cartridge 1500 to be described below. In the present embodiment, it is assumed that the staple link assembly 1170 includes one link member 1171, and the link member 1171 includes a first link 1172 and a second link 1173.

The first link 1172 is formed in the form of an elongated bar, which may have through holes formed at both end portions. The link coupling part 1161b of the staple pulley 1161 may be inserted through the through hole at one end portion of the first link 1172. The second link 1173 may be inserted through the through hole at another end portion of the first link 1172.

The second link 1173 is formed in the form of an elongated bar, and may be coupled to the first link 1172. The second link 1173 may include a first protrusion 1173a, a second protrusion 1173b, and a coupling part 1173c.

In detail, the first protrusion 1173a may be formed at one end portion of the second link 1173. The first protrusion 1173a is axially coupled to the first link 1172 by being fitted into the through hole of the first link 1172, so that the second link 1173 may be coupled to the first link 1172. In addition, the first protrusion 1173a may be fitted into a guide groove 1101b of the first jaw 1101, which will be described below.

Meanwhile, the second protrusion 1173b may be formed in one region of a central portion of the second link 1173. The second protrusion 1173b may be fitted into the guide groove 1101b of the first jaw 1101, which will be described below.

As described above, as the first protrusion 1173a and the second protrusion 1173b are moved along the guide groove 1101b in a state in which the first protrusion 1173a and the second protrusion 1173b of the second link 1173 formed in a protruding shape are fitted into the groove-shaped guide groove 1101b, the staple link assembly 1170 is moved with respect to the first jaw 1101 (and the cartridge 1500 therein). This will be described in more detail later.

Meanwhile, the coupling part 1173c may be formed at another end portion of the second link 1173. The coupling part 1173c may be coupled to the moving member 1550 of the cartridge 1500, which will be described below.

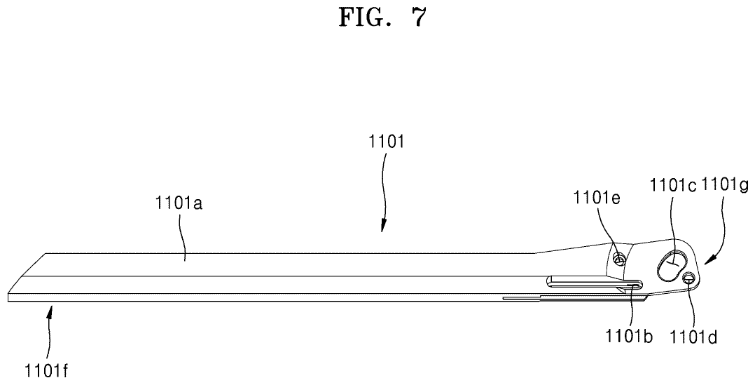

In the state as shown in FIG. 6, when the staple pulley 1161 is rotated in the clockwise direction, the link member 1171 connected to the staple pulley 1161 may be moved as a whole toward a distal end (see 1101f in FIG. 7) of the first jaw 1101. In contrast, when the staple pulley 1161 is rotated in the counterclockwise direction, the link member 1171 connected to the staple pulley 1161 may be moved as a whole toward a proximal end (see 1101g in FIG. 7) of the first jaw 1101.

Thus, a bidirectional rotational motion of the staple pulley assembly 1160 causes a reciprocating linear motion of the moving member 1550 of the cartridge 1500 through the staple link assembly 1170. This will be described in more detail later.

### Motion of First and Second Jaws

FIG. 7 is a plan view illustrating the first jaw of the end tool of the surgical instrument of FIG. 3.

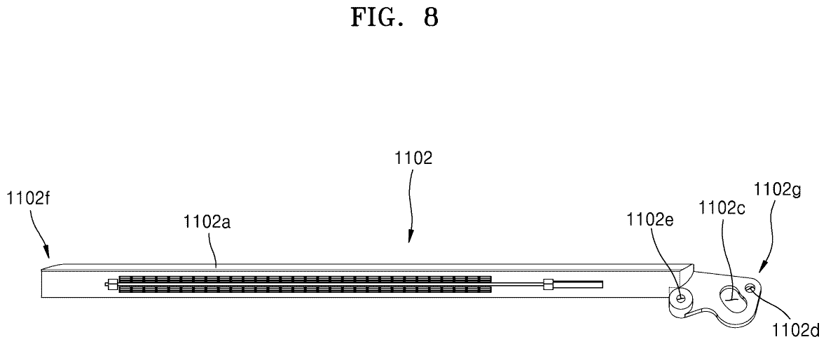

FIG. 8 is a plan view illustrating the second jaw of the end tool of the surgical instrument of FIG. 3.

Referring to FIGS. 7 and 8 and the like, the first jaw 1101 includes a cartridge accommodation part 1101a, the guide groove 1101b, a movable-coupling hole 1101c, a jaw pulley coupling hole 1101d, and a shaft pass-through part 1101e.

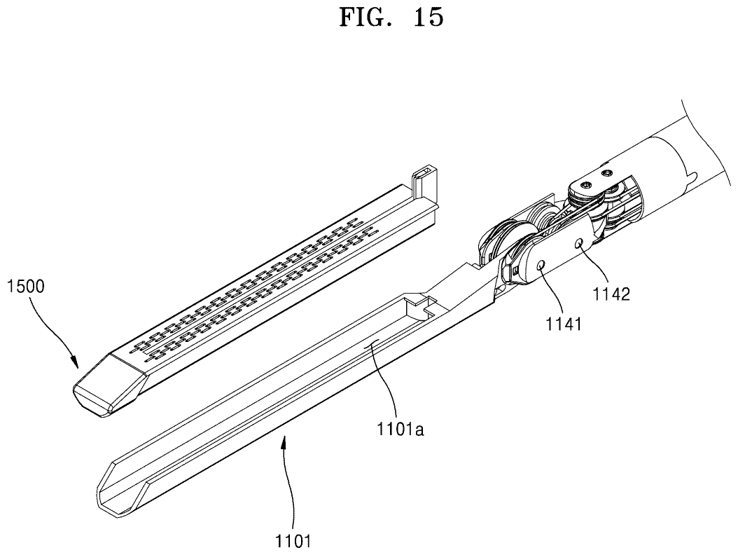

The first jaw 1101 is formed entirely in the shape of an elongated bar, the cartridge (1500 in FIG. 15) is accommodated in the distal end 1101f side, and the pulley 1111 is coupled to the proximal end 1101g, so that the first jaw 1101 is formed to be rotatable around the rotation shaft 1141. In other words, the first jaw 1101 may be formed entirely in the form of a hollow box with one surface (upper surface) thereof removed, such that the cartridge accommodation part 1101a capable of accommodating the cartridge 1500 may be formed inside the first jaw 1101. That is, the first jaw 1101 may be formed in a substantially “U” shape in cross section.

The guide groove 1101b configured to guide the movement of the staple link assembly 1170, which will be described below, may be formed on one side of the cartridge accommodation part 1101a of the first jaw 1101, e.g., on the proximal end 1101g side. The guide groove 1101b may be formed in the shape of a groove formed along a moving path of the staple link assembly 1170. In addition, as the first protrusion 1173a and the second protrusion 1173b are moved along the guide groove 1101b in a state in which the first protrusion 1173a and the second protrusion 1173b of the second link 1173 formed in a protruding shape are fitted into the groove-shaped guide groove 1101b, the staple link assembly 1170 is moved with respect to the first jaw 1101 (and the cartridge 1500 therein). That is, the staple link assembly 1170 may be moved along the guide groove 1101b of the first jaw 1101.

Meanwhile, the movable-coupling hole 1101c, the jaw pulley coupling hole 1101d, and the shaft pass-through part 1101e may be formed on the proximal end side of the first jaw 1101.

Here, the movable-coupling hole 1101c may be formed to have a predetermined curvature, and may be formed in an approximately elliptical shape. A shaft coupling part of the pulley 1111 may be fitted into the movable-coupling hole 1101c. Here, a short radius of the movable-coupling hole 1101c may be formed to be substantially the same as or slightly greater than a radius of the shaft coupling part. Meanwhile, a long radius of the movable-coupling hole 1101c may be formed to be greater than the radius of the shaft coupling part. Thus, in a state in which the shaft coupling part of the pulley 1111 is fitted into the movable-coupling hole 1101c of the first jaw 1101, the shaft coupling part is movable to a certain extent in the movable-coupling hole 1101c.

Meanwhile, the jaw pulley coupling hole 1101d is formed in the form of a cylindrical hole, and a jaw coupling part of the pulley 1111 may be fitted into the jaw pulley coupling hole 1101d. Here, a radius of the jaw pulley coupling hole 1101d may be formed to be substantially the same as or slightly greater than a radius of the jaw coupling part. Thus, the jaw coupling part of the pulley 1111 may be formed to be rotatably coupled to the jaw pulley coupling hole 1101d of the first jaw 1101.

The shaft pass-through part 1101e may be formed at the distal end 1101f side of the first jaw 1101 relative to the movable-coupling hole 1101c and the jaw pulley coupling hole 1101d. The shaft pass-through part 1101e may be formed in the form of a hole, and the jaw rotation shaft may be inserted through the shaft pass-through part 1101e.

The second jaw 1102 includes an anvil 1102a, a movable-coupling hole 1102c, a jaw pulley coupling hole 1102d, and a shaft pass-through part 1102e.

The second jaw 1102 is formed entirely in the shape of an elongated bar, the anvil 1102a is formed on a distal end 1102f side, and the pulley 1112 is coupled to a proximal end 1102g, so that the second jaw 1102 is formed to be rotatable around the rotation shaft 1141.

In detail, the anvil 1102a is formed in the form of a flat plane, on one surface of which shapes corresponding to the shapes of staples 1530 to be described below may be formed. The above-described anvil 1102a may serve as a support for supporting the staple 1530 on the opposite side of the operation member 1540 when the operation member 1540 pushes and raises the staple 1530 during a stapling motion, so that the staple 1530 is bent.

Meanwhile, the movable-coupling hole 1102c, the jaw pulley coupling hole 1102d, and the shaft pass-through part 1102e may be formed on the proximal end side of the second jaw 1102.

Here, the movable-coupling hole 1102c may be formed to have a predetermined curvature, and may be formed in an approximately elliptical shape. A shaft coupling part of the pulley 1121 may be fitted into the movable-coupling hole 1102c. Here, a short radius of the movable-coupling hole 1102c may be formed to be substantially the same as or slightly greater than a radius of the shaft coupling part. Meanwhile, a long radius of the movable-coupling hole 1102c may be formed to be greater than the radius of the shaft coupling part. Thus, in a state in which the shaft coupling part of the pulley 1121 is fitted into the movable-coupling hole 1102c of the second jaw 1102, the shaft coupling part is movable to a certain extent in the movable-coupling hole 1102c.

Meanwhile, the jaw pulley coupling hole 1102d is formed in the form of a cylindrical hole, and a jaw coupling part of the pulley 1121 may be fitted into the jaw pulley coupling hole 1102d. Here, a radius of the jaw pulley coupling hole 1102d may be formed to be substantially the same as or slightly greater than a radius of the jaw coupling part. Thus, the jaw coupling part of the pulley 1121 may be formed to be rotatably coupled to the jaw pulley coupling hole 1102d of the second jaw 1102.

Meanwhile, the shaft pass-through part 1102e may be formed at the proximal end 1102g side of the second jaw 1102 relative to the movable-coupling hole 1102c and the jaw pulley coupling hole 1102d. The shaft pass-through part 1102e may be formed in the form of a hole, and the jaw rotation shaft may be inserted through the shaft pass-through part 1102e.

The coupling relationship between the components described above is as follows.

The rotation shaft 1141, which is an end tool jaw pulley rotation shaft, is sequentially inserted through the shaft coupling part of the pulley 1111, the movable-coupling hole 1101c of the first jaw 1101, the shaft pass-through part 1161a of the staple pulley 1161, the movable-coupling hole 1102c of the second jaw 1102, and a shaft coupling part 1121a of the pulley 1121.

The jaw rotation shaft is sequentially inserted through the shaft pass-through part 1101e of the first jaw 1101 and the shaft pass-through part 1102e of the second jaw 1102.

The shaft coupling part of the pulley 1111 is fitted into the movable-coupling hole 1101c of the first jaw 1101, and the jaw coupling part of the pulley 1111 is fitted into the jaw pulley coupling hole 1101d of the first jaw 1101.

At this time, the jaw pulley coupling hole 1101d of the first jaw 1101 and the jaw coupling part of the pulley 1111 are axially coupled to each other so as to be rotatable, and the movable-coupling hole 1101c of the first jaw 1101 and the shaft coupling part of the pulley 1111 are movably coupled to each other.

The shaft coupling part 1121a of the pulley 1121 is fitted into the movable-coupling hole 1102c of the second jaw 1102, and a jaw coupling part 1121b of the pulley 1121 is fitted into the jaw pulley coupling hole 1102d of the second jaw 1102.

At this time, the jaw pulley coupling hole 1102d of the second jaw 1102 and the jaw coupling part 1121b of the pulley 1121 are axially coupled to each other to be rotatable, and the movable-coupling hole 1102c of the second jaw 1102 and the shaft coupling part 1121a of the pulley 1121 are movably coupled to each other.

Here, the pulley 1111 and the pulley 1121 are rotated around the rotation shaft 1141, which is an end tool jaw pulley rotation shaft. The first jaw 1101 and the second jaw 1102 are rotated around the jaw rotation shaft. That is, the pulley 1111 and the first jaw 1101 have different shafts of rotation. Similarly, the pulley 1121 and the second jaw 1102 have different shafts of rotation.

That is, the rotation angle of the first jaw 1101 is limited to a certain extent by the movable-coupling hole 1101c, but the first jaw 1101 is essentially rotated around a rotation shaft 1145, which is a jaw rotation shaft. Similarly, the rotation angle of the second jaw 1102 is limited to a certain extent by the movable-coupling hole 1102c, but the second jaw 1102 is essentially rotated around the rotation shaft 1145, which is a jaw rotation shaft.

This allows a grip force to be amplified. That is, in the surgical instrument 1000 according to an embodiment of the present disclosure, the coupling structure of the first jaw 1101 and the second jaw 1102 forms an X-shaped structure, and thus, when the first jaw 1101 and the second jaw 1102 are rotated in directions close to each other (i.e., when the first jaw 1101 and the second jaw 1102 are closed), the grip force is greater in a direction in which the first jaw 1101 and the second jaw 1102 are closed. This will be described below in more detail.

As described above, in motions of the first jaw 1101 and the second jaw 1102 being opened and closed, there are two axes that serve as the centers of rotation for the first jaw 1101 and the second jaw 1102. That is, the first jaw 1101 and the second jaw 1102 perform opening and closing motions around two axes of the rotation shaft 1141 and a shaft that is different from the rotation shaft 1141.

Meanwhile, although not shown in the drawings, in an optional embodiment, the first jaw 1101 and the second jaw 1102 may perform a motion around one rotation shaft, and for example, the jaw pulley rotation shaft and the jaw rotation shaft may be the same.

### Modified Example of Staple Drive Assembly

Hereinafter, one modified example of the staple drive assembly (1150 in FIG. 6 or the like) of the surgical instrument 1000 of FIG. 1 will be described.

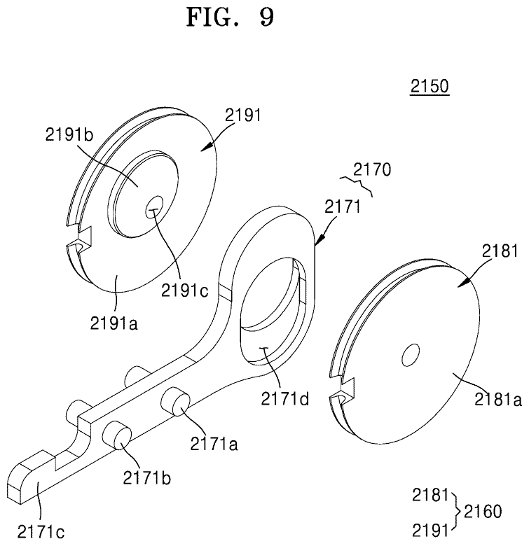

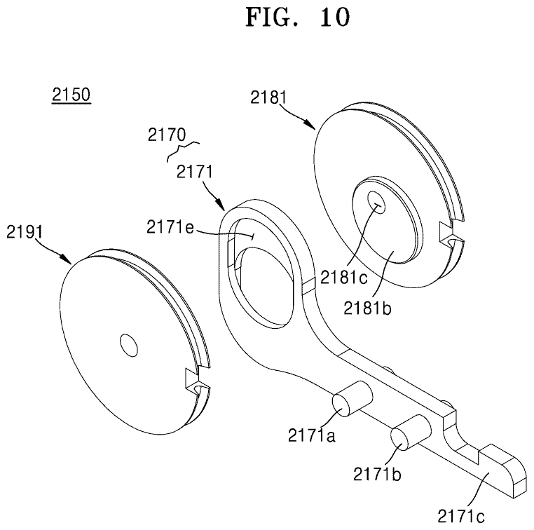

FIGS. 9 and 10 are exploded perspective views illustrating one modified example of the staple drive assembly of the surgical instrument of FIG. 1.

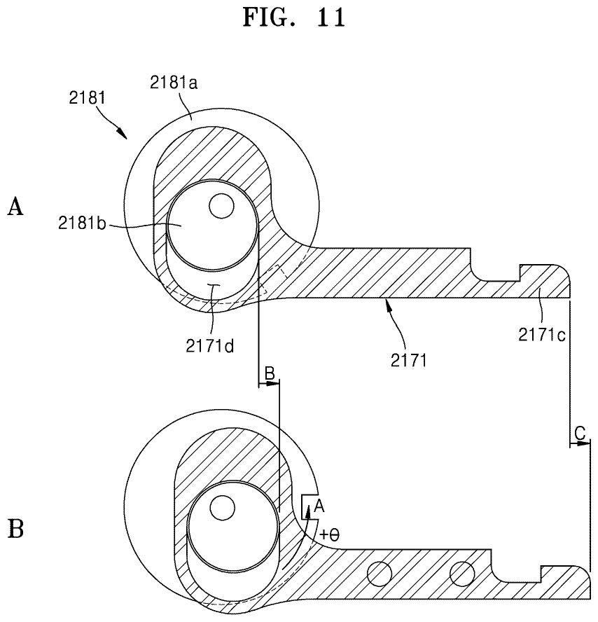

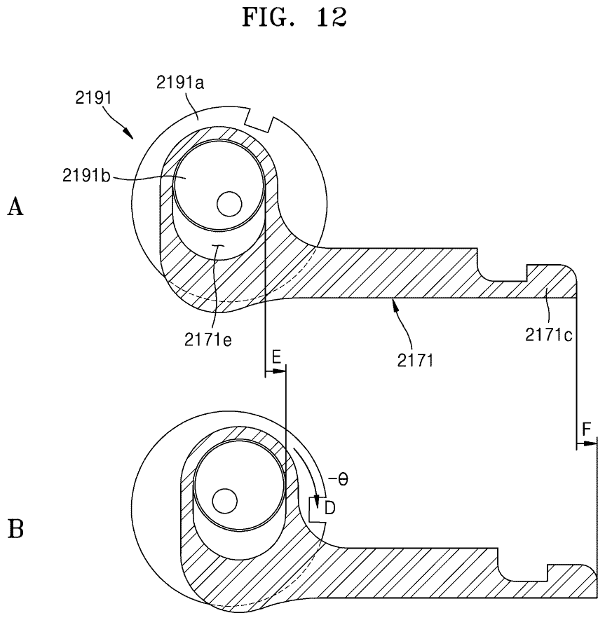

FIGS. 11 and 12 are side views illustrating one modified example of the staple drive assembly of the surgical instrument of FIG. 1.

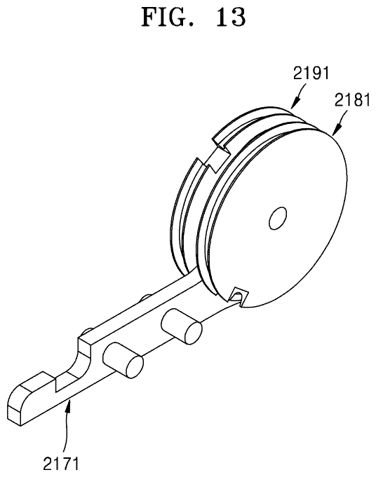

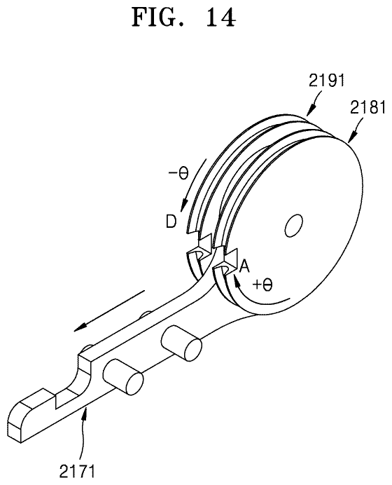

FIGS. 13 and 14 are perspective views illustrating operations of the staple drive assembly of FIGS. 9 to 12.

Referring to FIGS. 9 to 14, a staple drive assembly 2150 may include a staple pulley assembly 2160 and a staple link assembly 2170. Here, the staple drive assembly 2150 is connected to the moving member 1550 of the cartridge 1500 described above, and converts a rotational motion of the staple pulley assembly 2160 into a linear motion of the moving member 1550.

The staple pulley assembly 2160 may include one or more staple pulleys. In detail, the staple pulley assembly 2160 may include a first staple pulley 2181 and a second staple pulley 2191.

The staple link assembly 2170 may include one or more link members 2171. In addition, the link member 2171 may include one or more links. For example, in the present modified example, it is assumed that the staple link assembly 2170 includes one link member 2171, and the link member 2171 includes one link.

In the present modified example, the staple pulley assembly 2160 and the staple link assembly 2170 form a cam-slot structure. And with this structure, the effect of amplifying the force that moves the moving member 1550 forward can be obtained.

In detail, the staple pulley assembly 2160 may include the first staple pulley 2181 and the second staple pulley 2191.

The first staple pulley 2181 may include a body 2181a, a protruding member 2181b, and a shaft pass-through part 2181c.

The body 2181a is formed in the shape of a disk.

The shaft pass-through part 2181c may be formed in a center portion of the body 2181a. The shaft pass-through part 2181c may be formed in the form of a hole, and the end tool jaw pulley rotation shaft may be inserted through the shaft pass-through part 2181c.

In addition, the protruding member 2181b may be formed on the body 2181a of the first staple pulley 2181. The protruding member 2181b may be coupled to the link member 2171 of the staple link assembly 2170. Here, the center of the protruding member 2181b may not coincide with the center of the first staple pulley 2181, and the protruding member 2181b may be formed to be eccentric to a certain extent with respect to the first staple pulley 2181. The protruding member 2181b may be fitted into a first slot 2171d of the link member 2171, which will be described below.

The second staple pulley 2191 may include a body 2191a, a protruding member 2191b, and a shaft pass-through part 2191c.

The body 2191a is formed in the shape of a disk.

The shaft pass-through part 2191c may be formed in a center portion of the body 2191a. The shaft pass-through part 2191c may be formed in the form of a hole, and the end tool jaw pulley rotation shaft may be inserted through the shaft pass-through part 2191c.

In addition, the protruding member 2191b may be formed on the body 2191a of the second staple pulley 2191. The protruding member 2191b may be coupled to the link member 2171 of the staple link assembly 2170. Here, the center of the protruding member 2191b may not coincide with the center of the second staple pulley 2191, and the protruding member 2191b may be formed to be eccentric to a certain extent with respect to the second staple pulley 2191. The protruding member 2191b may be fitted into a second slot 2171e of the link member 2171, which will be described below.

Meanwhile, the end tool 1100 of the present disclosure may further include the staple link assembly 2170 connected to the staple pulley assembly 2160, and the staple link assembly 2170 may include the link member 2171. Here, the staple link assembly 2170 may serve to connect the staple pulley assembly 2160 to the moving member 1550 of the cartridge 1500 to be described below.

In the present embodiment, the staple link assembly 2170 includes one link member 2171, and the link member 2171 includes only one link. That is, by coupling the staple pulley assembly 2160 and the staple link assembly 2170 by a cam-slot structure, it is possible to convert a rotational motion of the staple pulley assembly 2160 into a linear motion of the staple link assembly 2170 even when the staple link assembly 2170 includes only one link.

In detail, the link member 2171 may be formed as a single link.

The link member 2171 is formed in a shape in which an elongated bar and an elliptical-shaped flat plate are coupled, and may have a bent portion, for example, formed approximately in the shape of the alphabet letter “L.” Here, the link member 2171 may include a first protrusion 2171a, a second protrusion 2171b, a coupling part 2171c, the first slot 2171d, and the second slot 2171e.

The first protrusion 2171a and the second protrusion 2171b may be formed in one region of a central portion of the link member 2171. The first protrusion 2171a and the second protrusion 2171b may be fitted into the guide groove (1101b in FIG. 7) of the first jaw.

As described above, as the first protrusion 2171a and the second protrusion 2171b are moved along the guide groove 1101b in a state in which the first protrusion 2171a and the second protrusion 2171b of the link member 2171 formed in a protruding shape are fitted into the groove-shaped guide groove 1101b, the link member 2171 is moved with respect to the first jaw 1101 (and the cartridge 1500 therein). This will be described in more detail later.

Meanwhile, the coupling part 2171c may be formed at one end portion of the link member 2171. The coupling part 2171c may be coupled to the moving member 1550 of the cartridge 1500.

Meanwhile, the first slot 2171d and the second slot 2171e may be formed at an end portion of the link member 2171 opposite to the one end portion at which the coupling part 2171c is formed.

In detail, the first slot 2171d may be formed in a surface of the link member 2171 facing the first staple pulley 2181. Here, the first slot 2171d is formed in the form of an elongated hole, into which the protruding member 2181b of the first staple pulley 2181 may be fitted. The first slot 2171d is formed to have a predetermined curvature, and may be formed in an approximately elliptical shape. At this time, the first slot 2171d may be formed to be larger than the protruding member 2181b by a certain extent. Thus, the protruding member 2181b is formed to be movable in the first slot 2171d by a certain extent in a state in which the protruding member 2181b of the first staple pulley 2181 is fitted into the first slot 2171d of the link member 2171.

As described above, the protruding member 2181b may be formed to be eccentric to a certain extent with respect to the center of the first staple pulley 2181. Thus, when the first staple pulley 2181 is rotated, the protruding member 2181b, while in contact with the first slot 2171d, may push the first slot 2171d to move the link member 2171. That is, when the first staple pulley 2181 is rotated, the protruding member 2181b, while in contact with the first slot 2171d, is moved in the first slot 2171d, which causes the link member 2171 to be linearly moved along the guide groove 1101b of the first jaw 1101.

Here, the first slot 2171d may be formed not to pass through the entire thickness of the link member 2171 but to pass through approximately half of the entire thickness of the link member 2171. In other words, the first slot 2171d may be formed to have substantially the same thickness as the thickness of the protruding member 2181b of the first staple pulley 2181.

Meanwhile, the second slot 2171e may be formed in the link member 2171. In detail, the second slot 2171e may be formed in a surface of the link member 2171 facing the second staple pulley 2191. Here, the second slot 2171e is formed in the form of an elongated hole, into which the protruding member 2191b of the second staple pulley 2191 may be fitted. The second slot 2171e is formed to have a predetermined curvature, and may be formed in an approximately elliptical shape. At this time, the second slot 2171e may be formed to be larger than the protruding member 2191b by a certain extent. Thus, the protruding member 2191b is formed to be movable in the second slot 2171e by a certain extent in a state in which the protruding member 2191b of the second staple pulley 2191 is fitted into the second slot 2171e of the link member 2171.

As described above, the protruding member 2191b may be formed to be eccentric to a certain extent with respect to the center of the second staple pulley 2191. Thus, when the second staple pulley 2191 is rotated, the protruding member 2191b, while in contact with the second slot 2171e, may push the second slot 2171e to move the link member 2171. That is, when the second staple pulley 2191 is rotated, the protruding member 2191b, while in contact with the second slot 2171e, is moved in the second slot 2171e, which causes the link member 2171 to be linearly moved along the guide groove 1101b of the first jaw (1101 in FIG. 7).

Here, the second slot 2171e may be formed not to pass through the entire thickness of the link member 2171 but to pass through approximately half of the entire thickness of the link member 2171. In other words, the second slot 2171e may be formed to have substantially the same thickness as the thickness of the protruding member 2191b of the second staple pulley 2191.

Here, the first slot 2171d and the second slot 2171e may be formed to at least partially overlap. In addition, the sum of the thicknesses of the first slot 2171d and the second slot 2171e in the Y-axis direction may be formed to be approximately equal to the thickness of the link member 2171 in the Y-axis direction.

Here, the first slot 2171d and the second slot 2171e may be formed to be vertically symmetrical with respect to the rotation shaft. As such, since the first slot 2171d and the second slot 2171e are formed to be vertically symmetrical with respect to the rotation shaft, the protruding member 2181b of the first staple pulley 2181 coupled to the link member 2171 and the protruding member 2191b of the second staple pulley 2191 may be disposed to be symmetrical to each other. This will be described in more detail later.

### Displacement and Motion of Staple Link Assembly According to Rotation of Staple Pulley

Hereinafter, displacement of the staple link assembly 2170 according to the rotation of the first staple pulley 2181 and the second staple pulley 2191 will be described.

Referring to FIG. 11, the first staple pulley 2181 and the staple link assembly 2170 are coupled in the form of a cam-slot That is, the cam-shaped protruding member 2181b formed on the first staple pulley 2181 is coupled to the first slot 2171d formed in the link member 2171. Accordingly, when the first staple pulley 2181 is rotated in the direction of an arrow A, the displacement of the protruding member 2181b of the first staple pulley 2181 in the X-axis direction becomes B. In addition, a displacement of the staple link assembly 2170 in the X-axis direction becomes C.

Similarly, referring to FIG. 12, in the present modified example, the second staple pulley 2191 and the staple link assembly 2170 are coupled in the form of a cam-slot. That is, the cam-shaped protruding member 2191b formed on the second staple pulley 2191 is coupled to the second slot 2171e formed in the link member 2171. Accordingly, when the second staple pulley 2191 is rotated in the direction of an arrow D, the displacement of the protruding member 2191b of the second staple pulley 2191 in the X-axis direction becomes E. In addition, the displacement of the staple link assembly 2170 in the X-axis direction becomes F.

When the staple pulley and the staple link assembly are coupled in a link-shaft manner rather than a cam-slot manner as compared with the above case, the displacement of the staple link assembly in the X-axis direction will be much increased.

In other words, as compared to the case in which the staple pulley and the staple link assembly are axially coupled, when the staple pulley and the staple link assembly are cam-slot coupled as in the present modified example, the displacement of the staple link assembly in the X-axis direction will be reduced even when the staple pulley is rotated by the same amount.

Meanwhile, since work is the product of force and displacement, assuming that the work for rotating the staple pulley is the same, the displacement and the force are inversely proportional to each other. Accordingly, when the displacement is reduced, the force is increased in inverse proportion to the displacement.

As a result, in the present modified example, each of the first staple pulley 2181 and the second staple pulley 2191 is coupled in the form of a cam-slot to the staple link assembly 2170, and the displacement of the staple link assembly 2170 in the X-axis direction caused by the rotation of the first staple pulley 2181 and the second staple pulley 2191 is relatively reduced as compared to the embodiment described above, and thus the force received by the staple link assembly 2170 in the X-axis direction is increased relative to a simple link structure.

According to the present modified example described above, a force for moving the staple link assembly 2170 and the moving member 1550 connected thereto forward is amplified, and thus a stapling motion may be performed more robustly.

In particular, in the present modified example, since two staple pulleys (i.e., the first staple pulley 2181 and the second staple pulley 2191) that are symmetrical to each other are provided, a force with which the staple pulley assembly 2160 pushes the staple link assembly 2170 may be amplified approximately twice as compared to when only one staple pulley is provided.

In addition, the first staple pulley 2181 and the second staple pulley 2191 are symmetrically disposed in a left and right direction with respect to the X Z plane, and thus balanced in the left and right direction in performing a stapling motion, so that the end tool stably performs motions with respect to the yaw rotation shaft without moving in the left and right direction as a whole. In addition, by making winding directions of the wire corresponding to the first staple pulley 2181 and the wire corresponding to the second staple pulley 2191 opposite to each other, movements with respect to the rotation shaft 1143 may be mutually offset.

Hereinafter, rotation directions of the first staple pulley 2181 and the second staple pulley 2191 will be described.

Referring to FIGS. 11 to 14, the staple link assembly 2170 is moved forward when the first staple pulley 2181 rotates in the direction of an arrow A in FIG. 14 (i.e., in the clockwise direction), and the staple link assembly 2170 is moved forward when the second staple pulley 2191 rotates in the direction of an arrow D in FIG. 14 (i.e., in the counterclockwise direction).

In contrast, the first staple pulley 2181 moves the staple link assembly 2170 backward when rotated in the counterclockwise direction, and the second staple pulley 2191 moves the staple link assembly 2170 backward when rotated in the clockwise direction.

As a result, when the first staple pulley 2181 and the second staple pulley 2191 are rotated in opposite directions, the staple link assembly 2170 is moved (forward or backward). In contrast, when the first staple pulley 2181 and the second staple pulley 2191 are rotated in the same direction, the rotations of the two pulleys are canceled out and thus the staple link assembly 2170 is not moved.

As a result, in the state as shown in FIG. 13, when the first staple pulley 2181 is rotated in the clockwise direction while the second staple pulley 2191 is rotated in the counterclockwise direction, the link member 2171 connected to the first staple pulley 2181 and the second staple pulley 2191 may be moved as a whole toward the distal end (1101f in FIG. 7) of the first jaw (1101 in FIG. 7).

In contrast, when the first staple pulley 2181 is rotated in the counterclockwise direction while the second staple pulley 2191 is rotated in the clockwise direction, the link member 2171 connected to the first staple pulley 2181 and the second staple pulley 2191 may be moved as a whole toward the proximal end (1101g in FIG. 7) of the first jaw (1101 in FIG. 7).

Thus, a bidirectional rotational motion of the staple pulley assembly 2160 causes a reciprocating linear motion of the moving member 1550 of the cartridge 1500 through the staple link assembly 2170.

The driving part 1208 may simultaneously drive the first staple pulley 2181 and the second staple pulley 2191 of the staple pulley assembly 2160. For example, the driving part 1208 may drive the first staple pulley 2181 and the second staple pulley 2191 by reversing the driving of two wires through an intermediate conversion member such as a slide member or the like from the rotational motion of the motor member.

### Cartridge

Hereinafter, the cartridge 1500 of the surgical instrument 1000 of FIG. 1 will be described in more detail.

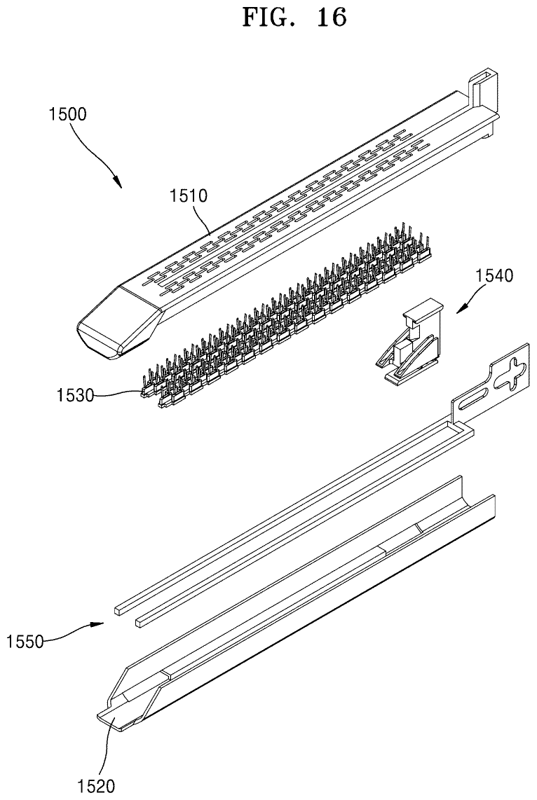

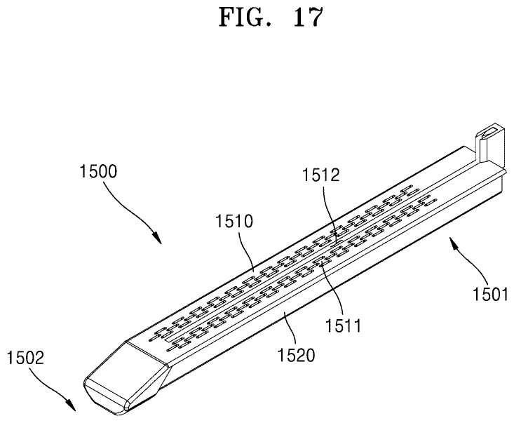

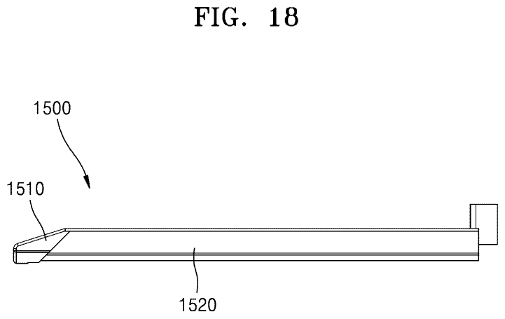

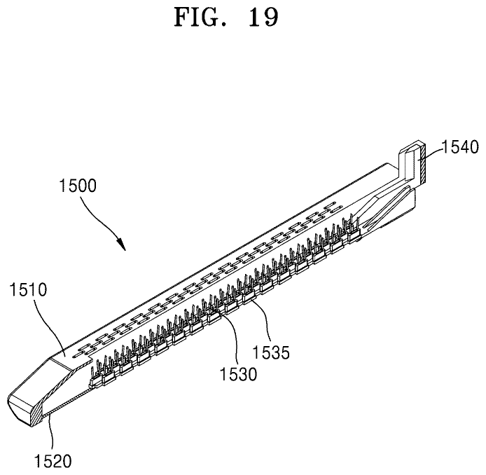

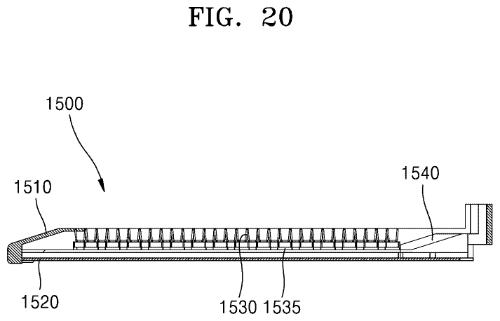

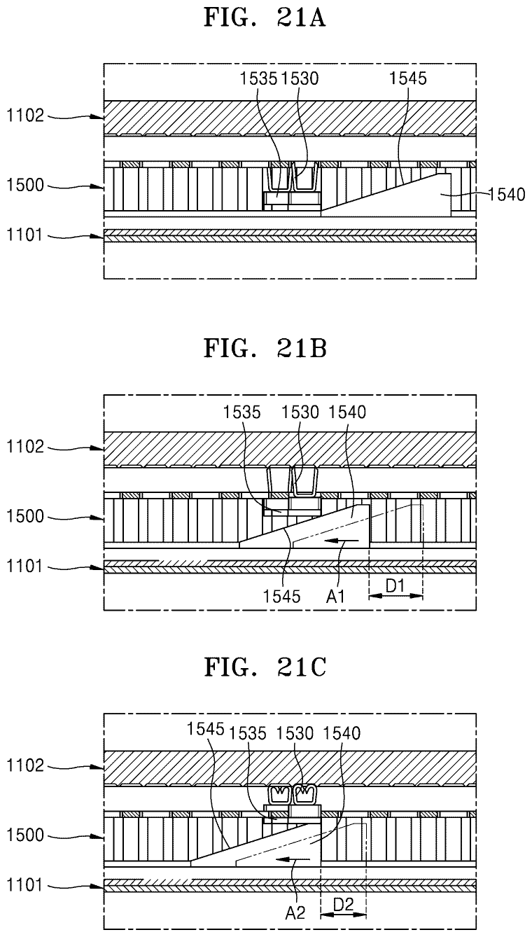

FIG. 15 is a perspective view illustrating the first jaw and the cartridge of the surgical instrument of FIG. 1. FIG. 16 is an exploded perspective view of the cartridge of FIG. 15. FIG. 17 is a coupled perspective view of the cartridge of FIG. 15. FIG. 18 is a side view of the cartridge of FIG. 15. FIG. 19 is a perspective cross-sectional view for describing an internal structure of the cartridge of FIG. 15. FIG. 20 is a side cross-sectional view for describing the internal structure of the cartridge of FIG. 15. FIGS. 21A to 21C are views for describing a staple motion of the end tool of FIG. 3.

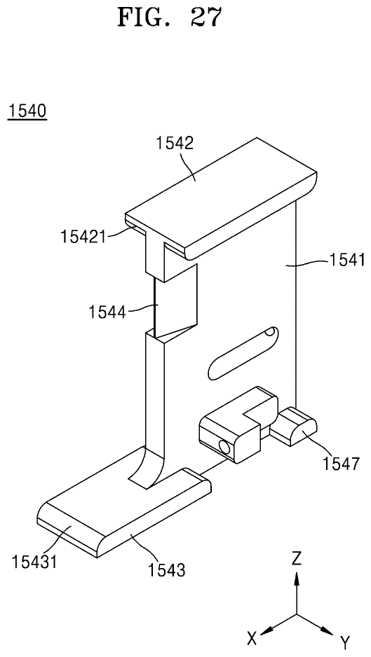

Referring to FIGS. 15 to 21C and the like, the cartridge 1500 is formed to be mountable to and dismountable from the first jaw 1101, and includes a plurality of staples 1530 and a blade (1544 in FIG. 27) therein to perform suturing and cutting tissue. Here, the cartridge 1500 may include a cover 1510, a housing 1520, the staples 1530, withdrawal members 1535, the operation member 1540, and the moving member 1550.

The housing 1520 forms an outer shape of the cartridge 1500, and may be formed entirely in the form of a hollow box with one surface (upper surface) thereof removed to accommodate the moving member 1550, the operation member 1540, and the staple 1530 therein. Here, the housing 1520 may be formed in an approximately “U” shape in cross section.

The cover 1510 is formed to cover an upper portion of the housing 1520. Staple holes 1511 through which the plurality of staples 1530 may be ejected to the outside may be formed in the cover 1510. As the staples 1530, which are accommodated inside the housing 1520 before a stapling operation, are pushed and raised upward by the operation member 1540 during a stapling motion, and pass through the staple holes 1511 of the cover 1510 to be withdrawn to the outside of the cartridge 1500, stapling is performed.

Meanwhile, a slit 1512 may be formed in the cover 1510 along a length direction of the cover 1510. The blade (1542 in FIG. 27) of the operation member 1540 may protrude out of the cartridge 1500 through the slit 1512. As the blade (1544 in FIG. 27) of the operation member 1540 passes along the slit 1512, staple-completed tissue may be cut.

The plurality of staples 1530 may be disposed inside the housing 1520. As the operation member 1540, which will be described below, is linearly moved in one direction, the plurality of staples 1530 are sequentially pushed and raised from the inside of the housing 1520 to the outside, thereby performing suturing, that is, stapling. Here, the staples 1530 may be made of a material that may include titanium, stainless steel, or the like.

Meanwhile, the withdrawal member 1535 may be further disposed between the housing 1520 and the staple 1530. In other words, it may be described that the staple 1530 is disposed above the withdrawal member 1535. In this case, the operation member 1540 is linearly moved in one direction to push and raise the withdrawal member 1535, and the withdrawal member 1535 may push and raise the staple 1530.

As such, the operation member 1540 may be described as pushing and raising the staples 1530 in both the case in which the operation member 1540 directly pushes and raises the staples 1530 and the case in which the operation member 1540 pushes and raises the withdrawal members 1535 and the withdrawal members 1535 pushes and raises the staples 1530 (i.e., the operation member 1540 indirectly pushes and raises the staples 1530).

The moving member 1550 may be disposed at an inner lower side of the housing 1520.

Here, the moving member 1550 is not fixedly coupled to the other components of the cartridge 1500, and may be formed to be movable relative to the other components of the cartridge 1500. That is, the moving member 1550 may perform a reciprocating linear motion with respect to the housing 1520 and the cover 1510 coupled to the housing 1520.

The operation member 1540 may be disposed inside the housing 1520. The operation member 1540 is formed to be in contact with the moving member 1550, and may be formed to linearly move in one direction in response to a reciprocating linear motion of the moving member 1550. In other words, the operation member 1540 interacts with the moving member 1550 to perform stapling and cutting motions while moving in the extension direction of the connection part 1400.

The configuration of the operation member 1540 and the moving member 1550 will be described in detail below.

The cartridge 1500 is accommodated in the cartridge accommodation part 1101a of the first jaw 1101, and in this case, the moving member 1550 of the cartridge 1500 is coupled to the staple link assembly 1170 of the end tool 1100. Accordingly, the rotational motion of the staple pulley 1161 of the end tool 1100 is converted into a linear motion of the moving member 1550 through the staple link assembly 1170.

In this case, when the moving member 1550 is connected to the staple pulley 1161 through the staple link assembly 1170, and the staple pulley 1161 is rotated alternately in the clockwise/counterclockwise directions, the moving member 1550 may be repeatedly moved forward and backward. In addition, when the moving member 1550 is moved forward, the operation member 1540 may be moved forward together with the moving member 1550, and when the moving member 1550 is moved backward, only the moving member 1550 may be moved backward and the operation member 1540 may remain stationary in place. As the operation member 1540 is moved forward while repeating this process, the staple 1530 may be stapled by a wedge 1545 while the blade (1544 in FIG. 27) cuts stapled tissue.

This will be described in more detail as follows.

### Stapling and Cutting Motions

When the driving force is transmitted to the staple pulley 1161 through a driving transmission part such as a wire by the driving part 1208, the staple pulley 1161 is rotated in one of the clockwise direction and the counterclockwise direction, and the staple link assembly 1170 connected to the staple pulley 1161 and the moving member 1550 of the cartridge 1500 connected to the staple link assembly 1170 are moved toward a distal end 1502 of the cartridge 1500.

In addition, when the moving member 1550 is moved toward the distal end 1502 of the cartridge 1500, the operation member 1540 in contact with the moving member 1550 is moved toward the distal end 1502 of the cartridge 1500 together with the moving member 1550.

In addition, as the operation member 1540 is moved toward the distal end 1502 of the cartridge 1500, the blade (1544 in FIG. 27) of the operation member 1540 is moved toward the distal end 1502 of the cartridge 1500 while the operation member 1540 ejects the staples 1530 out of the cartridge 1500.

Meanwhile, when the staple pulley 1161 is rotated in another one of the clockwise direction and the counterclockwise direction, the staple link assembly 1170 connected to the staple pulley 1161 and the moving member 1550 of the cartridge 1500 connected to the staple link assembly 1170 are moved toward a proximal end 1501 of the cartridge 1500, and in this case, the operation member 1540 is stationary.

In addition, as the above-described operations are repeatedly performed, a stapling motion by the wedge 1545 and a cutting motion by the blade (1544 in FIG. 27) are simultaneously performed.

FIGS. 21A to 21C are views for describing a staple motion of the end tool of FIG. 3.

Referring to FIGS. 21A to 21C, in the state as shown in FIG. 21A, as the operation member 1540 is moved in the direction of an arrow A1 of FIG. 21B, the wedge 1545 of the operation member 1540 pushes and raises the withdrawal member 1535, and the withdrawal member 1535 pushes and raises one side of a lower portion of the staple 1530. In addition, due thereto, the staple 1530 is ejected to the outside of the first jaw 1101 and the cartridge 1500.

In this state, when the operation member 1540 is further moved in the direction of an arrow A2 of FIG. 21C, the ejected staple 1530 is continuously pushed and raised by the operation member 1540 while in contact with the anvil 1102a of the second jaw 1102, so that stapling is performed while both end portions of the staple 1530 are bent.

As such motions are continuously performed, stapling is sequentially performed in the plurality of staples 1530 from the staple 1530 at the proximal end 1501 side to the staple 1530 at the distal end 1502 side.

### Correlation Between Stapling and Cutting Motions and Other Motions

Hereinafter, a correlation between stapling and cutting motions and other motions (pitch, yaw, and actuation motions) will be described.

First, when the end tool 1100 performs a pitch motion, the staple pulley 1161 also performs a pitch motion. That is, when the pulley 1111 and the pulley 1121 perform pitch motions of being rotated in the same direction around the rotation shaft 1143, the staple pulley 1161 should also be rotated in the same direction as the pulley 1111 and the pulley 1121. If the staple pulley 1161 is not rotated together with the pulley 1111 and the pulley 1121 when the pulley 1111 and the pulley 1121 are rotated around the rotation shaft 1143, there is a risk that the cartridge 1500 connected to the staple pulley 1161 is moved relative to the first jaw 1101 and is disconnected from the first jaw 1101. Further, rotation of the staple pulley 1161 that is not synchronized with that of the pulley 1111 may cause the moving member 1550 to unintentionally move forward, which in turn may cause an unintended stapling motion.

Next, when the end tool 1100 performs a yaw motion, the staple pulley 1161 also performs a yaw motion. That is, when the pulley 1111 and the pulley 1121 perform yaw motions of being rotated in the same direction around the rotation shaft 1141, the staple pulley 1161 should also be rotated in the same direction as the pulley 1111 and the pulley 1121. If the staple pulley 1161 is not rotated together with the pulley 1111 and the pulley 1121 when the pulley 1111 and the pulley 1121 are rotated around the rotation shaft 1141, there is a risk that the cartridge 1500 connected to the staple pulley 1161 is moved relative to the first jaw 1101 and is disconnected from the first jaw 1101. Further, rotation of the staple pulley 1161 that is not synchronized with that of the pulley 1111 may cause the moving member 1550 to unintentionally move forward, which in turn may cause an unintended stapling motion.

Next, when the end tool 1100 performs an actuation motion, the staple pulley 1161 is rotated together with the pulley 1111. That is, when the pulley 1111 and the pulley 1121 perform actuation motions of being rotated in opposite directions around the rotation shaft 1141, the staple pulley 1161 should be rotated in the same direction as the pulley 1111. If the staple pulley 1161 is not rotated together with the pulley 1111 when the pulley 1111 is rotated around the rotation shaft 1143, there is a risk that the cartridge 1500 connected to the staple pulley 1161 is moved relative to the first jaw 1101 and is disconnected from the first jaw 1101. Further, rotation of the staple pulley 1161 that is not synchronized with that of the pulley 1111 may cause the moving member 1550 to unintentionally move forward, which in turn may cause an unintended stapling motion.

Meanwhile, when the end tool 1100 performs stapling and cutting motions, the pulley 1111 and the pulley 1121 are not rotated. That is, when the staple pulley 1161 is rotated around the rotation shaft 1141 and the moving member 1550 of the link member 1171 and the cartridge 1500 connected thereto performs a linear reciprocating motion, the pulley 1111 and the pulley 1121 should not be rotated. Otherwise, the first jaw 1101 or the second jaw 1102 is rotated during the stapling and cutting motion, and the stapling and cutting motions will not be performed normally.

As a result, when the pulley 1111, which is a first jaw pulley, is rotated, the staple pulley 1161 accommodated in the first jaw 1101 should be also rotated together with the pulley 1111. On the other hand, when the staple pulley 1161 is rotated for the stapling and cutting motions, the pulley 1111 and the pulley 1121 should be formed to maintain positions thereof without rotating. As such, the correlation between the stapling and cutting motions and other motions (the yaw and actuation motions) has been discussed above.

In other words, the pulley 1111 and the pulley 1121 may be said to be independent of the rotation of the staple pulley 1161. That is, even when the staple pulley 1161 is rotated by the staple wire, the pulley 1111 and the pulley 1121 may not be rotated. In contrast, the staple pulley 1161 may be said to be dependent of the rotation of the pulley 1111. That is, when the pulley 1111 is rotated by the jaw wire, the staple pulley 1161 may be formed to be rotated together with the pulley 1111.

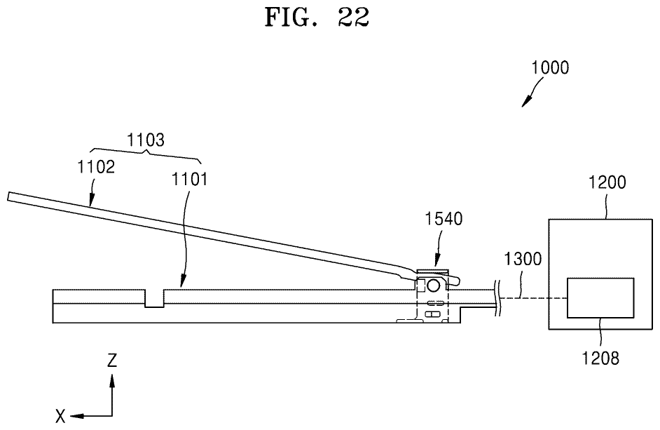

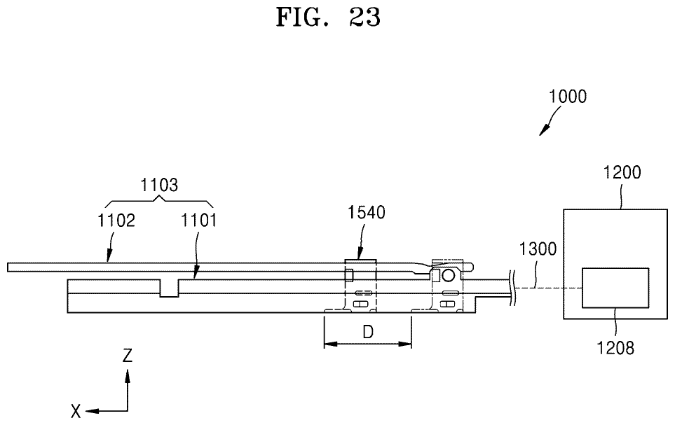

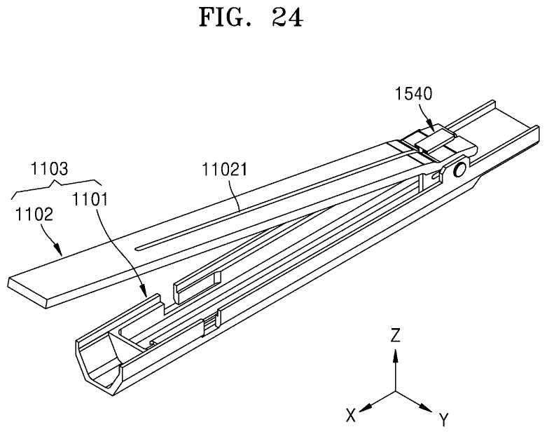

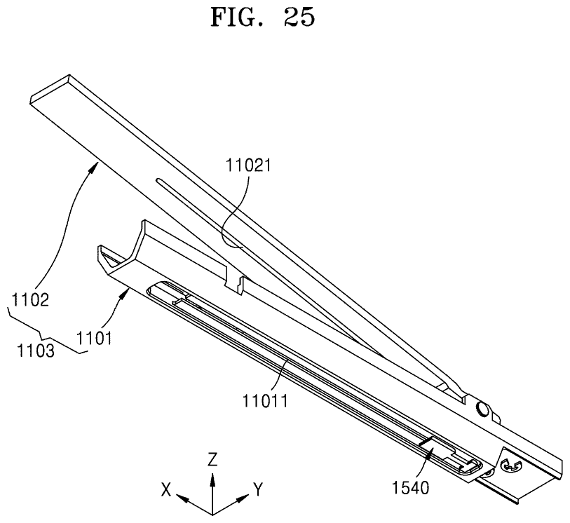

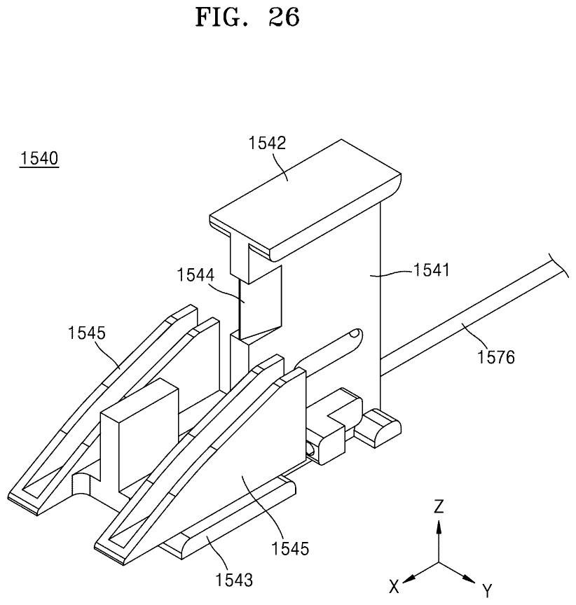

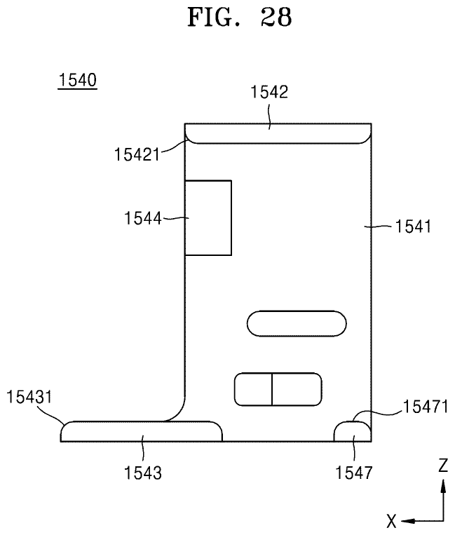

FIG. 22 is a view schematically illustrating the surgical instrument 1000 according to an embodiment of the present disclosure, and FIG. 23 is a view for describing operations of the operation member 1540 of the surgical instrument 1000 of FIG. 22. FIGS. 24 and 25 are perspective views illustrating the jaws 1103 of FIG. 22, FIG. 26 is a perspective view illustrating the operation member 1540 of FIG. 23, FIG. 27 is a view with the wedge 1545 omitted from FIG. 26, and FIG. 28 is a side view of FIG. 27.

First, referring to FIGS. 22 to 25, the surgical instrument 1000 according to an embodiment of the present disclosure may include the end tool 1100. The end tool 1100 may be manipulated through the manipulation part 1200.

The end tool 1100 is disposed on one side of the surgical instrument 1000, and may be inserted into a surgical site to perform motions necessary for surgery. For example, the end tool 1100 may be configured to rotate in at least one direction.

The end tool 1100 may function as a surgical clamp, grasper, vessel sealer, or stapler, but the present disclosure is not limited thereto, and any device that is inserted into a patient's surgical site and performs a motion necessary for surgery may be used as the end tool 1100.

Hereinafter, for convenience of description, the embodiment in which the end tool 100 is used as a stapler will be mainly described.

The end tool 1100 may be connected to the manipulation part 1200 through the connection part 1400. As described above, one or more wires and electric wires may be accommodated in the connection part 1400. Accordingly, when a user manipulates the manipulation part 1200, the end tool 1100 may be operated by the user's manipulation.

The end tool 1100 may include the jaw 1103.

The jaw 1103 may be in various forms. For example, the jaw 1103 may be configured to form a set of at least one or more jaws. As an exemplary example, the jaw 1103 may consist of a pair of jaws 1103, including the first jaw 1101 and the second jaw 1102.

Depending on the function of the end tool 1100, the first jaw 1101 and the second jaw 1102 may be formed in the same shape or in different shapes.

The first jaw 1101 and the second jaw 1102 may be disposed to face each other. For example, the first jaw 1101 and the second jaw 1102 may be rotatably connected to each other in one region.

At least one of the first jaw 1101 and the second jaw 1102 may be rotatable. Specifically, referring to FIGS. 22 and 23, the second jaw 1102 may be rotatable with respect to the first jaw 1101. In other words, the first jaw 1101 and the second jaw 1102 may be connected to each other by a rotation shaft in one region, and the second jaw 1102 may be rotated around the rotation shaft in a direction of spreading apart or converging toward the first jaw 1101. By such a rotational motion, the first jaw 1101 and the second jaw 1102 may perform a grip motion or a clamping motion.

The first jaw 1101 may be formed to extend to a length. For example, the first jaw 1101 may be formed in the shape of an elongated bar, with a length having a value greater than a value of a width thereof.

The cartridge accommodation part (1101a in FIG. 15) may be provided inside the first jaw 1101. The cartridge 1500 may be accommodated in the cartridge accommodation part (1101a in FIG. 15). The cartridge accommodation part (1101a in FIG. 15) may be formed to be open on one surface, for example, the surface facing the second jaw 1102 may be formed to be open. In addition, the operation member 1540 may be disposed in the cartridge accommodation part (1101a in FIG. 15).

The operation member 1540 may be moved while being accommodated in the cartridge accommodation part (1101a in FIG. 15). For example, the operation member 1540 may be moved in a longitudinal direction of the jaw 1103 while being accommodated in the cartridge accommodation part (1101a in FIG. 15).

The second jaw 1102 may be formed to extend to a length. For example, the second jaw 1102 may be formed in the shape of an elongated bar, with a length having a value greater than a value of a width thereof.

The operation member 1540 may be moved in the longitudinal direction of the jaw 1103 in a state in which the first jaw 1101 and the second jaw 1102 are closed (in a state in which the first jaw 1101 and the second jaw 1102 are facing each other). In other words, the operation member 1540 may be accommodated in the first jaw 1101, with one side connected to the first jaw 1101 and another side connected to the second jaw 1102. Accordingly, the operation member 1540 may apply a force on the first jaw 1101 and the second jaw 1102 while moving from the proximal end of the jaw 1103 toward the distal end of the jaw 1103, causing the first jaw 1101 and the second jaw 1102 to close to each other. Thereafter, after the first jaw 1101 and the second jaw 1102 are closed to each other, the operation member 1540 may be moved in the longitudinal direction of the first jaw 1101 and the second jaw 1102, specifically, in a direction toward the distal end of each of the first jaw 1101 and the second jaw 1102. In other words, the operation member 1540 may apply a force to the first jaw 1101 and the second jaw 1102 while moving in a direction from the proximal end of the jaw 1103 toward the distal end of the jaw 1103, causing the first jaw 1101 and the second jaw 1102 to perform a clamping motion.

In an embodiment, the anvil 1102a may be formed on one surface of the second jaw 1102. For example, the anvil 1102a may be formed in the second jaw 1102 at a portion facing the first jaw 1101 (a portion facing the staple accommodated in the first jaw 1101).

As the operation member 1540 moves, the end tool 1100 of the surgical instrument 1000 may perform one or more motions. For example, as the operation member 1540 moves, the staple (not shown) accommodated in the first jaw 1101 may be withdrawn toward the second jaw 1102, thereby performing stapling.

Referring to FIGS. 22 and 23 again, the operation member 1540 may apply a force to the jaws 1103 in a direction in a direction in which the first jaw 1101 and the second jaw 1102 are closed to each other, by initiating movement from the state of FIG. 22. Thereafter, the operation member 1540 may be linearly moved a certain distance D from the state shown in FIG. 23. For example, the operation member 1540 may be moved forward and/or backward in the longitudinal direction of the jaw 1103.

As described above, as the operation member 1540 moves, the wedge 1545 formed on the operation member 1540 pushes and raises the staple or withdrawal member as will be described below, so that the staple motion of the end tool 1100 may be implemented.

In an embodiment, although not shown in the drawings, the end tool 1100 may further include a stopper (not shown). The stopper (not shown) may define the movement limit of the operation member 1540. In other words, the stopper (not shown) may be disposed on a path through which the operation member 1540 is moved toward the distal end of the jaw 1103, and when the operation member 1540 is moved beyond a certain distance, the stopper may block the path, preventing further forward movement of the operation member 1540.

Although not shown in the drawings, the operation member 1540 may receive a driving force from a separately provided member.

As an example, the operation member 1540 may receive a driving force through a separately provided moving member (not shown). For example, the moving member (not shown) may be formed to move linearly in one or both directions.

The moving member (not shown) may be disposed in one region of the end tool 1100 and, for example, may be disposed to be at least partially accommodated inside the first jaw 1101. The moving member (not shown) may be at least partially in contact with or connected to the operation member 1540, and may move the operation member 1540 while being moved by a driving force source, as will be described below.

The moving member (not shown) may be moved by various driving force sources, for example, by the driving part 1208. The driving part 1208 includes a driving force source, and may be formed in various forms, such as a motor or actuator.

However, the present disclosure is not limited thereto, and the moving member (not shown) may be formed to have various structures, for example, any component capable of moving the operation member 1540 by receiving a driving force may be used.

The driving part 1208 may be disposed in one region of the manipulation part 1200. In addition, in another optional embodiment, the driving part 1208 may be disposed in another region of the surgical instrument 1000, e.g., in one region of the end tool 1100, one region of the connection part 1400 that connects the end tool 1100 to the manipulation part 1200, or one region outside the surgical instrument 1000.

The driving part 1208 may be connected to the moving member (not shown) by one or more power transmission parts 1300, and a driving force of the driving part 1208, e.g., a rotating force, may be transmitted to the moving member (not shown) through the power transmission parts 1300. As a specific example, the power transmission parts 1300 may include one or more wires.

A rotational force of the driving part 1208 may be transmitted to the moving member (not shown) through the power transmission parts 1300 including one or more wires. In an optional embodiment, the driving part 1208 may be connected to the power transmission parts 1300 through one or more pulleys.

A backward movement wire 1576 may be connected to one side of the operation member 1540. For example, the backward movement wire 1576 may be connected to a rear surface (a proximal end 1540b) of the operation member 1540.

The backward movement wire 1576 may transmit a driving force to the operation member 1540 to move the operation member 1540 backward. That is, the operation member 1540 may perform a backward movement through the backward movement wire 1576. In other words, the backward movement of the operation member 1540 through the backward movement wire 1576 may be performed by pulling the operation member 1540 through the backward movement wire 1576.

Although not shown in the drawings, the driving part 1208 or the driving transmission part (e.g., wires, pulleys, and the like) capable of pulling the backward movement wire 1576 may be connected to the backward movement wire 1576, and the backward movement wire 1576 may be operated according to manual or automatic manipulation.

Hereinafter, the operation member 1540 according to an embodiment of the present disclosure will be described in detail with reference to FIGS. 26 to 28.

### Operation Member

As described above, the operation member 1540 may perform an operation (clamping operation) of closing the jaws 1103 while moving in the longitudinal direction of the jaw 1103 and an operation of linearly moving in the longitudinal direction of the jaw 1103.

The operation member 1540 may include a body 1541 and at least one or more flanges.

The body 1541 is a member that forms an overall outer shape of the operation member 1540. The body 1541 may be formed in a substantially elongated rectangular shape.

The flanges may be formed at both ends of the body 1541 to prevent the operation member 1540 from separating from the jaw 1103.

The operation member 1540 may include a first flange 1542.

The first flange 1542 may be disposed on one side of the body 1541 and formed to have a width greater than that of the body 1541. In other words, when viewing the operation member 1540 in a direction from the distal end of the jaw 1103 toward the proximal end, i.e., in the X-axis direction, the first flange 1542 may be formed to be transversely wider than the body 1541.

The first flange 1542 may be formed to extend to a length in the X-axis direction of FIG. 27. In an embodiment, the first flange 1542 may be formed to extend to a length greater than or equal to a length of the body 1541 (the length of the body 1541 in the X-axis direction of FIG. 27).

The first flange 1542 may serve to prevent the operation member 1540 from separating from the jaw 1103 while moving along the jaw 1103.

Specifically, the second jaw 1102 may include a second slit 11021 that is formed to extend in a longitudinal direction of the second jaw 1102. At this time, the operation member 1540 may move with at least a portion of the body 1541 accommodated in the second slit 11021. In this case, the first flange 1542 may be disposed above the second jaw 1102, and the body 1541 may be disposed to pass through the second slit 11021 from the first flange 1542. In other words, the first flange 1542 may be caught on an upper surface of the second jaw 1102, and the body 1541 may extend in a direction passing through the second slit 11021 from the first flange 1542.

In an optional embodiment, the first flange 1542 may include a first flange curved part 15421 that is formed to be curved. For example, the portion of the first flange 1542 in contact with the second jaw 1102 may be formed to be curved. Accordingly, when the operation member 1540 is moved in a state in which the first flange 1542 and the second jaw 1102 are in contact with each other, a resistance force generated between the first flange 1542 and the second jaw 1102 may be reduced.

The operation member 1540 may include a second flange 1543.

The second flange 1543 may be disposed on another side of the body 1541. That is, the second flange 1543 may be disposed on the side of the body 1541 in the opposite direction to the first flange 1542. The second flange 1543 may be formed to have a width greater than that of the body 1541. In other words, when viewing the operation member 1540 in the direction from the distal end of the jaw 1103 toward the proximal end, i.e., in the X-axis direction, the second flange 1543 may be formed to be transversely wider than the body 1541.

The second flange 1543 may be formed to extend to a length in the X-axis direction of FIG. 27.

The second flange 1543 may serve to prevent the operation member 1540 from separating from the jaw 1103 while moving along the jaw 1103.

Specifically, the first jaw 1101 may include a first slit 11011 that is formed to extend in a longitudinal direction of the first jaw 1101. At this time, the operation member 1540 may move with at least a portion of the body 1541 accommodated in the first slit 11011. In this case, the second flange 1543 may be disposed below the first jaw 1101, and the body 1541 may be disposed to pass through the first slit 11011 from the second flange 1543. In other words, the second flange 1543 may be caught on a lower surface of the first jaw 1101, and the body 1541 may extend in a direction passing through the first slit 11011 from the second flange 1543.

In an optional embodiment, the second flange 1543 may include a second flange curved part 15431 that is formed to be curved. For example, the portion of the second flange 1543 in contact with the first jaw 1101 may be formed to be curved. Accordingly, when the operation member 1540 is moved in a state in which the second flange 1543 and the first jaw 1101 are in contact with each other, a resistance force generated between the second flange 1543 and the first jaw 1101 may be reduced.

Meanwhile, the first flange 1542 and the second flange 1543 may come into contact with the first jaw 1101 and the second jaw 1102, respectively, during a clamping operation of the operation member 1540, as will be described below, to transmit a force so that the first jaw 1101 and the second jaw 1102 are closed.

At least a portion of the first flange 1542 may come into contact with the second jaw 1102 when the operation member 1540 is moved in the longitudinal direction of the jaw 1103.

As an example, when the operation member 1540 is moved in the longitudinal direction of the jaw 1103, a portion of the first flange 1542 may come into contact with the second jaw 1102, while the remaining portion may not be in contact with the second jaw 1102. In other words, it may be described that when the operation member 1540 is moved in the longitudinal direction of the jaw 1103, the first flange 1542 may come into line contact with the second jaw 1102 or a portion of the first flange 1542 may come into surface contact with the second jaw 1102.

As another example, when the operation member 1540 is moved in the longitudinal direction of the jaw 1103, the entire area of the first flange 1542 may come into contact with the second jaw 1102. In other words, when the operation member 1540 is moved in the longitudinal direction of the jaw 1103, the first flange 1542 may come into surface contact with the second jaw 1102 across the entire area of the first flange 1542.

In addition, at least a portion of the second flange 1543 may come into contact with the first jaw 1101 when the operation member 1540 is moved in the longitudinal direction of the jaw 1103.

As an example, when the operation member 1540 is moved in the longitudinal direction of the jaw 1103, a portion of the second flange 1543 may come into contact with the first jaw 1101, while the remaining portion may not be in contact with the first jaw 1101. In other words, it may be described that when the operation member 1540 is moved in the longitudinal direction of the jaw 1103, the second flange 1543 may come into line contact with the first jaw 1101 or a portion of the second flange 1543 may come into surface contact with the first jaw 1101.

As another example, when the operation member 1540 is moved in the longitudinal direction of the jaw 1103, the entire area of the second flange 1543 may come into contact with the first jaw 1101. In other words, when the operation member 1540 is moved in the longitudinal direction of the jaw 1103, the second flange 1543 may come into surface contact with the first jaw 1101 across the entire area of the second flange 1543.

In other words, the operation member 1540 may be subjected to a rotational force in at least one direction while moving along the jaw 1103. For example, while moving toward the distal end of the jaw 1103, the operation member 1540 may be subjected to a rotational force, causing the operation member 1540 to rotate in a direction that moves the second flange 1543 further forward toward the distal end of the jaw 1103 than the first flange 1542. In this case, the operation member 1540 may receive repulsive and frictional forces from the jaw 1103, and accordingly, the sum of rotational moments acting on the operation member 1540 becomes zero, preventing the operation member 1540 from rotating. That is, the first flange 1542 and the second flange 1543 move linearly while in contact with the jaws 1103, but do not rotate. In other words, the operation member 1540 is moved linearly, but not rotated, with the first flange 1542 and the second flange 1543 partially or fully in surface contact with the second jaw 1102 and the first jaw 1101.

In other words, the operation member 1540 may be formed such that a distance between the first flange 1542 and the second flange 1543 is greater than or equal to a vertical distance between the first jaw 1101 and the second jaw 1102. Specifically, a distance between a lower end surface of the first flange 1542 and an upper end surface of the second flange 1543 (refer to FIG. 28 or the like) may be greater than or equal to a distance between a surface of the first flange 1542 in contact with the second jaw 1102 and a surface of the second flange 1543 in contact with the first jaw 1101.

Thus, when a distance between the first flange 1542 and the second flange 1543 is equal to a distance between the first jaw 1101 and the second jaw 1102 when the operation member 1540 is connected to the first jaw 1101 and the second jaw 1102, the operation member 1540 can be linearly moved without rotating. Alternatively, when the distance between the first flange 1542 and the second flange 1543 is greater than the distance between the first jaw 1101 and the second jaw 1102, the operation member 1540 may be linearly moved after at least partially rotating.

In an optional embodiment, the operation member 1540 may further include a third flange 1547.

The third flange 1547 may be disposed on another side of the body 1541. In addition, the third flange 1547 may be formed parallel to the second flange 1543 and may be disposed on the proximal end 1540b of the operation member 1540. That is, based on FIG. 28, the first flange 1542 may be formed on an upper side of the body 1541, the second flange 1543 may be formed on a lower left side of the body 1541, and the third flange 1547 may be disposed on a lower right side of the body 1541.

In other words, it may be expressed that the second flange 1543 and the third flange 1547 are formed parallel to each other, but a separation space is formed between the second flange 1543 and the third flange 1547.

In an optional embodiment, the third flange 1547 may include a third flange curved part 15471 that is formed to be curved. For example, the portion of the third flange 1547 in contact with the first jaw 1101 may be formed to be curved. Accordingly, when the operation member 1540 is moved in a state in which the third flange 1547 and the first jaw 1101 are in contact with each other, a resistance force generated between the third flange 1547 and the first jaw 1101 may be reduced.

As will be described below, when the operation member 1540 is moved in the longitudinal direction of the jaw 1103, the operation member 1540 rotates, resulting in only a portion of each of the first flange 1542, the second flange 1543, and the third flange 1547 being in contact with the jaws 1103. In other words, the portion of the operation member 1540 between the second flange 1543 and the third flange 1547 is not in contact with the jaw 1103. Thus, by separating the flange disposed on the lower side of the body 1541 into the second flange 1543 and the third flange 1547 (by forming an empty space between the second flange 1543 and the third flange 1547), the weight of the operation member 1540 may be reduced. In addition, by forming the separation space between the second flange 1543 and the third flange 1547, any possible friction or resistance force may be prevented from being generated in the portion between the second flange 1543 and the third flange 1547.

In summary, the body 1541 of the operation member 1540 may move in the longitudinal direction of the jaw 1103 while being accommodated in the first slit 11011 and the second slit 11021. At this time, the first flange 1542 may be disposed above the second jaw 1102, the second flange 1543 and the third flange 1547 may be disposed below the first jaw 1101, and the body 1541 be disposed to pass through both the first slit 11011 and the second slit 11021. In addition, a distance between the first flange 1542 and the second flange 1543 and between the first flange 1542 and the third flange 1547 may be formed to have a value greater than or equal to that of a vertical distance between the lowermost end surface of the first jaw 1101 and the uppermost end surface of the second jaw 1102 in a state in which the first jaw 1101 and the second jaw 1102 are closed. Specifically, as described above, the distance between the lower end surface of the first flange 1542 and the upper end surface of the second flange 1543 (refer to FIG. 28 or the like) may be greater than or equal to the distance between the surface of the first flange 1542 in contact with the second jaw 1102 and the surface of the second flange 1543 in contact with the first jaw 1101. In addition, a distance between the lower end surface of the first flange 1542 and an upper end surface of the third flange 1547 (refer to FIG. 28 or the like) may be greater than or equal to a distance between the surface of the first flange 1542 in contact with the second jaw 1102 and the surface of the third flange 1547 in contact with the first jaw 1101.

In an optional embodiment, a width of the body 1541 may be formed to be less than a width of each of the first slit 11011 and the second slit 11021. That is, the occurrence of unnecessary friction, which may be generated when the body 1541 and the first and second slits 11011 and 11011 come into close contact or contact each other as the operation member 1540 is moved in the longitudinal direction of the jaw 1103, may be prevented.

The operation member 1540 may further include the wedge 1545.

The wedge 1545 may be formed integrally with at least one side of the body 1541, or may be coupled to the body 1541 as a separate member.

The wedge 1545 may be formed to have a certain inclined surface. That is, the wedge 1545 may be formed to be inclined to a certain extent in the extension direction of the connection part 1400 (i.e., in the X-axis direction). In other words, the height of the wedge 1545 at the proximal end side of the cartridge may be formed to be greater than that at the distal end side. The wedge 1545 may be formed in various numbers and shapes depending on the shape of the staple or withdrawal member in contact therewith.

As such, the wedge 1545 may be formed to be sequentially in contact with the withdrawal members or the plurality of staples, and may serve to sequentially push and raise the staples. As shown in FIGS. 21A to 21C described above and elsewhere herein, the operation member 1540 may serve to withdraw the staples to the outside of the cartridge by sequentially pushing and raising the staples while moving toward the distal end.

The operation member 1540 may further include the blade 1544 formed on one side of the body 1541. For example, the blade 1544 may be formed on a distal end 1540a of the body 1541.

The blade 1544 may be sharply formed to be capable of cutting tissue.

That is, as at least a portion of the blade 1544 is withdrawn to the outside of the first jaw 1101 and the cartridge, tissue disposed between the first jaw 1101 and the second jaw 1102 may be cut.

As another example, the blade 1544 may always be withdrawn to the outside of the first jaw 1101. As another example, the blade 1544 may normally be accommodated inside the first jaw 1101 or inside the cartridge, and may be withdrawn to the outside of the first jaw 1101 only when the operation member 1540 is moved in a longitudinal direction.

### Modified Example of Operation Member

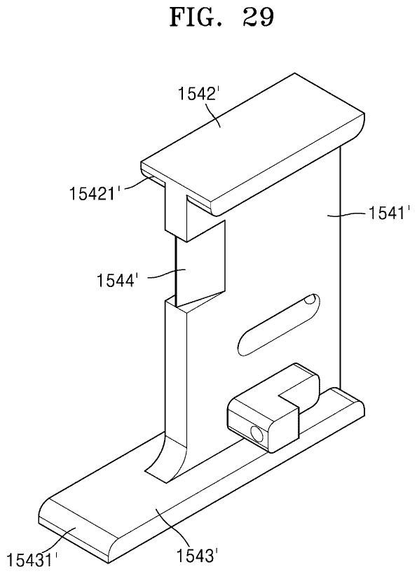

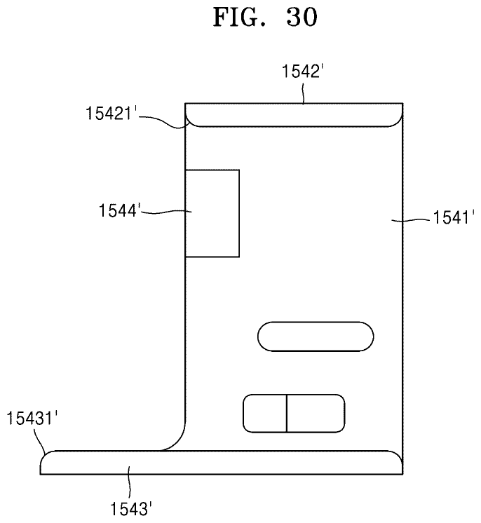

One modified example of the operation member of the present disclosure will be described with reference to FIGS. 29 and 30. Hereinafter, for convenience of description, the content that is the same as the operation member 1540 described with reference to FIGS. 26 to 28 or the content that may be easily changed and adopted by a person skilled in the art to which the present disclosure pertains will be omitted or briefly described.

FIG. 29 is a perspective view illustrating one modified example of the operation member of FIG. 27, and FIG. 30 is a side view of FIG. 29.

Referring to FIGS. 29 and 30, an operation member 1540′ according to the present embodiment is different from the operation member 1540 described above in that the second flange 1543 and the third flange 1547 are integrally formed.

The operation member 1540′ may include a body 1541′ and at least one or more flanges.

The body 1541′ is a member that forms an overall outer shape of the operation member 1540′. The body 1541′ may be formed in a substantially elongated rectangular shape.

The flanges may be formed at both ends of the body 1541′ to prevent the operation member 1540′ from separating from the jaw 1103.

The operation member 1540′ may include a first flange 1542′.

The first flange 1542′ may be disposed on one side of the body 1541′, above the second jaw 1102, and may be formed to have a width greater than that of the body 1541′. In other words, when viewing the operation member 1540′ in a direction from the distal end of the jaw 1103 toward the proximal end, i.e., in the X-axis direction, the first flange 1542′ may be formed to be transversely wider than the body 1541′.

Thus, since the operation member 1540′ moves with at least a portion of the body 1541′ accommodated in the second slit 11021 of the second jaw 1102, the first flange 1542′ may serve to prevent the operation member 1540′ from separating from the second jaw 1102 when the operation member 1540′ moves along the jaw 1103.

The first flange 1542′ may be formed to extend to a length (the length in the X-axis direction of FIG. 27). In an embodiment, the first flange 1542′ may be formed to extend to a length greater than or equal to a length of the body 1541′ (the length in the X-axis direction of FIG. 27).

In an optional embodiment, the first flange 1542′ may include a first flange curved part 15421′ that is formed to be curved. Accordingly, when the operation member 1540′ is moved in a state in which the first flange 1542′ and the second jaw 1102 are in contact with each other, a resistance force generated between the first flange 1542′ and the second jaw 1102 may be reduced.

The operation member 1540′ may include a second flange 1543′.

The second flange 1543′ may be disposed on another side of the body 1541′. That is, the second flange 1543′ may be disposed on the side of the first flange 1542′ in the opposite direction to the body 1541′, more specifically, the second flange 1543′ may be disposed below the first jaw 1101. The second flange 1543′ may be formed to have a width greater than that of the body 1541′. In other words, when viewing the operation member 1540′ in a direction from the distal end of the jaw 1103 toward the proximal end, i.e., in the X-axis direction, the second flange 1543′ may be formed to be transversely wider than the body 1541′.

Thus, since the operation member 1540′ moves with at least a portion of the body 1541′ accommodated in the first slit 11011 of the first jaw 1101, the second flange 1543′ may serve to prevent the operation member 1540′ from separating from the jaw 1103 when the operation member 1540′ moves along the jaw 1103.

The second flange 1543′ may be formed to extend to a length (the length in the X-axis direction of FIG. 27).

The second flange 1543′ according to the present embodiment may be formed to extend at least from a proximal end to a distal end of another side (a lower side surface based on FIG. 30) of the body 1541′. In other words, the second flange 1543′ may be formed to extend to a length greater than or equal to that of another side (the lower side surface based on FIG. 30) of the body 1541′. That is, it may be described that the second flange 1543′ is formed by connecting the second flange 1543 and the third flange 1547, compared to the embodiment of the operation member 1540 described with reference to FIGS. 26 to 28.

Accordingly, in describing the operation or the like of the operation member 1540′, the description of a region adjacent to the proximal end of the second flange 1543′ of the operation member 1540′ according to the present embodiment can be understood as referring to the third flange 1547, to the extent applicable.

In an optional embodiment, the second flange 1543′ may include a second flange curved part 15431′ that is formed to be curved. For example, the portion of the second flange 1543′ in contact with the first jaw 1101 may be formed to be curved. The second flange curved part 15431′ may be formed on both the distal end and the proximal end of the second flange 1543′, or on either side of the distal end and the proximal end. Accordingly, when the operation member 1540′ is moved in a state in which the second flange 1543′ and the first jaw 1101 are in contact with each other, a resistance force generated between the second flange 1543′ and the first jaw 1101 may be reduced.

As described above, the first flange 1542′ and the second flange 1543′ may come into contact with the first jaw 1101 and the second jaw 1102, respectively, during a clamping operation of the operation member 1540′ to transmit a force so that the first jaw 1101 and the second jaw 1102 are closed.

At least a portion or all of the first flange 1542′ may come into contact with the second jaw 1102 when the operation member 1540′ is moved in the longitudinal direction of the jaw 1103.

In addition, at least a portion or all of the second flange 1543′ may come into contact with the first jaw 1101 when the operation member 1540′ is moved in the longitudinal direction of the jaw 1103.

In other words, the operation member 1540′ may be subjected to a rotational force in at least one direction while moving along the jaw 1103. For example, while moving toward the distal end of the jaw 1103, the operation member 1540′ may be subjected to a rotational force, causing the operation member 1540′ to rotate in a direction that moves the second flange 1543′ further forward toward the distal end of the jaw 1103 than the first flange 1542′. In this case, the operation member 1540′ may receive repulsive and frictional forces from the jaw 1103, and accordingly, the sum of rotational moments acting on the operation member 1540′ becomes zero, preventing the operation member 1540′ from rotating.

In other words, the operation member 1540′ may be formed such that a distance between the first flange 1542′ and the second flange 1543′ is greater than or equal to a vertical distance between the first jaw 1101 and the second jaw 1102. Specifically, a distance between a lower end surface of the first flange 1542′ and an upper end surface of the second flange 1543′ (refer to FIG. 30 or the like) may be greater than or equal to a distance between a surface of the first flange 1542′ in contact with the second jaw 1102 and a surface of the second flange 1543′ in contact with the first jaw 1101.

Thus, when a distance between the first flange 1542′ and the second flange 1543′ is equal to a distance between the first jaw 1101 and the second jaw 1102 when the operation member 1540′ is connected to the first jaw 1101 and the second jaw 1102, the operation member 1540′ can be linearly moved without rotating. Alternatively, when the distance between the first flange 1542′ and the second flange 1543′ is greater than the distance between the first jaw 1101 and the second jaw 1102, the operation member 1540′ may be linearly moved after at least partially rotating.

In summary, the body 1541′ of the operation member 1540′ may move in the longitudinal direction of the jaw 1103 while being accommodated in the first slit 11011 and the second slit 11021. At this time, the first flange 1542′ may be disposed above the second jaw 1102, the second flange 1543′ may be disposed below the first jaw 1101, and the body 1541′ be disposed to pass through both the first slit 11011 and the second slit 11021. In addition, the distance between the second flange 1543′ and the second flange 1543′ may be formed to have a value greater than or equal to that of a vertical distance between the lowermost end surface of the first jaw 1101 and the uppermost end surface of the second jaw 1102 when the first jaw 1101 and the second jaw 1102 are closed. Specifically, as described above, the distance between the lower end surface of the first flange 1542′ and the upper end surface of the second flange 1543′ (refer to FIG. 30 or the like) may be greater than or equal to the distance between the surface of the first flange 1542′ in contact with the second jaw 1102 and the surface of the second flange 1543′ in contact with the first jaw 1101.

In an optional embodiment, a width of the body 1541′ may be formed to be less than a width of each of the first slit 11011 and the second slit 11021. That is, the occurrence of unnecessary friction, which may be generated when the body 1541′ and the first and second slits 11011 and 11011 come into close contact or contact each other as the operation member 1540′ is moved in the longitudinal direction of the jaw 1103, may be prevented.

The operation member 1540′ may further include a wedge 1545′.

The wedge 1545′ may be formed integrally with at least one side of the body 1541′, or may be coupled to the body 1541′ as a separate member.

The wedge 1545′ may be formed to have a certain inclined surface. That is, the wedge 1545′ may be formed to be inclined to a certain extent in the extension direction of the connection part 1400 (i.e., in the X-axis direction). In other words, the height of the wedge 1545′ at the proximal end side of the cartridge may be formed to be greater than that at the distal end side. The wedge 1545′ may be formed to be sequentially in contact with withdrawal members or a plurality of staples, and may serve to withdraw the staples to the outside of the cartridge by sequentially pushing and raising the staples while moving toward the distal end. The wedge 1545′ may be formed in various numbers and shapes depending on the shape of the staple or withdrawal member in contact therewith.

The operation member 1540′ may further include a blade 1544′ formed on one side of the body 1541′. For example, the blade 1544′ may be formed on a distal end 1540a′ of the body 1541′.

The blade 1544′ may be sharply formed to be capable of cutting tissue. That is, as at least a portion of the blade 1544′ is withdrawn to the outside of the first jaw 1101 and the cartridge, tissue disposed between the first jaw 1101 and the second jaw 1102 may be cut.

As another example, the blade 1544′ may always be withdrawn to the outside of the first jaw 1101. As another example, the blade 1544′ may normally be accommodated inside the first jaw 1101 or inside the cartridge, and may be withdrawn to the outside of the first jaw 1101 only when the operation member 1540′ is moved in a longitudinal direction.

### Operation of Operation Member

Hereinafter, operations of the operation member 1540 will be described in detail.

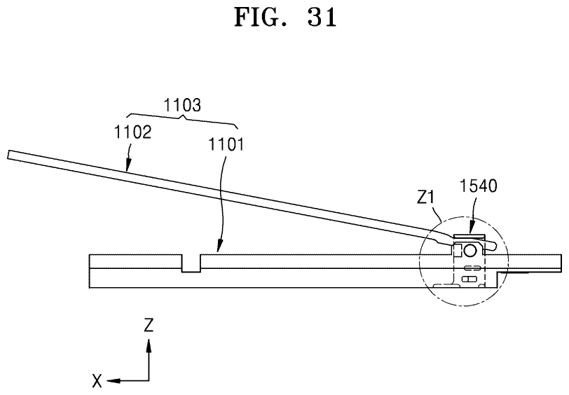

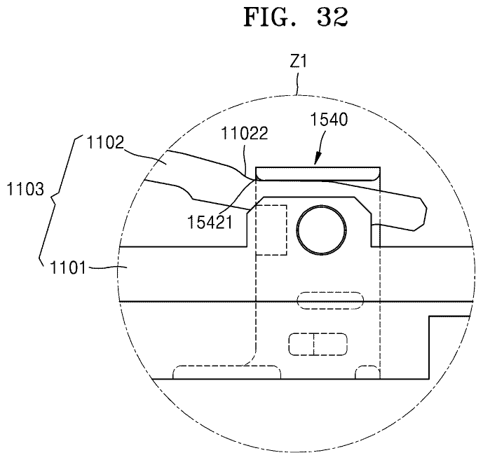

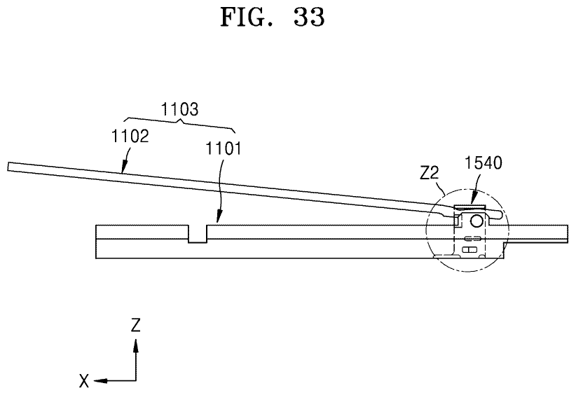

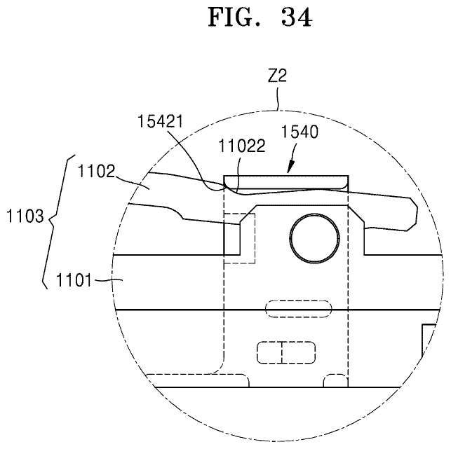

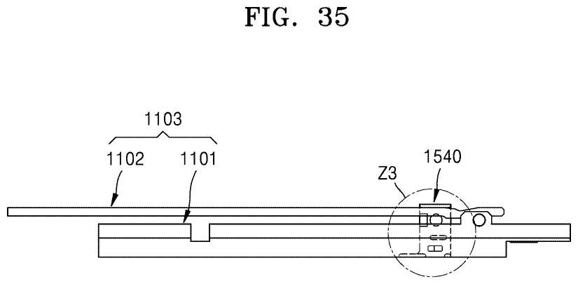

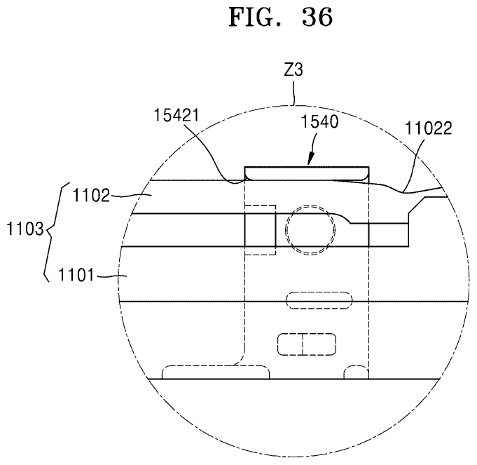

FIG. 31 is a view illustrating a state before the operation member 1540 of FIG. 23 performs an operation (clamping operation) of closing the jaws 1103, and FIG. 32 is an enlarged view of portion Z1 of FIG. 31. FIG. 33 is a view illustrating a state in which the operation member 1540 of FIG. 23 performs the operation (clamping operation) of closing the jaws 1103, and FIG. 34 is an enlarged view of portion Z2 of FIG. 33. FIG. 35 is a view illustrating a state after the operation member 1540 of FIG. 23 performs the operation (clamping operation) of closing the jaws 1103, and FIG. 36 is an enlarged view of portion Z3 of FIG. 35.

Referring to FIGS. 31 to 36, the second jaw 1102 may include a jaw-inclined surface 11022 formed in one region thereof. For example, the jaw-inclined surface 11022 may be formed at a position adjacent to the proximal end of the second jaw 1102.

The jaw-inclined surface 11022 may be formed to be inclined upward in a direction toward the distal end of the second jaw 1102. Specifically, the jaw-inclined surface 11022 may be formed to be partially inclined upward in the direction from the proximal end of the second jaw 1102 toward the distal end (in the X-axis direction), and then be flat (not inclined).

The jaw-inclined surface 11022 is the portion of the second jaw 1102, at which the first flange 1542 of the operation member 1540 comes into contact with the second jaw 1102 when the operation member 1540 begins to move toward the distal end of the jaw 1103 in a state in which the jaws 1103 are open (the state illustrated in FIG. 31). In other words, it may be said that the jaw-inclined surface 11022 is the portion of the second jaw 1102, to which the operation member 1540 applies a force to close (i.e., clamp) the open jaws 1103.

The jaw-inclined surface 11022 may be formed such that an inclination angle thereof varies in the longitudinal direction. This is to efficiently close the second jaw 1102 because an angle of rotation of the second jaw 1102 with respect to a moving distance of the operation member 1540, a force applied by the first flange 1542 on the jaw-inclined surface 11022, a value of rotational moment applied by the first flange 1542 on the jaw-inclined surface 11022, and the like are changed depending on the inclination angle at which the first flange 1542 of the operation member 1540 and the jaw-inclined surface 11022 are in contact with each other.

The inclination angle of the jaw-inclined surface 11022 at the proximal end may be formed to be greater than that at the distal end. In other words, it may be described that the jaw-inclined surface 11022 is steeply inclined upward at the proximal end and then gently inclined upward.

Describing this in more detail, when body tissue is located between the first jaw 1101 and the second jaw 1102, as an angle at which the body tissue is clamped is reduced, a greater force is required to clamp the body tissue. In other words, when the operation member 1540 performs a clamping operation while moving toward the distal end of the jaw 1103, a greater force must be applied to the jaw 1103 as the clamping process progresses compared to the initial stage.

Accordingly, the jaw-inclined surface 11022 according to the present disclosure is formed with a decreasing inclination angle toward the distal end of the jaw 1103 to increase a conversion rate of linear motion force toward the distal end of the operation member 1540 into a clamping force applied to the jaw 1103.

In an optional embodiment, the steeply upwardly inclined portion of the jaw-inclined surface 11022 may have an inclination angle of 60° to 90°. Thus, when the first flange 1542 of the operation member 1540 comes into contact with the proximal end of the jaw-inclined surface 11022 to close the second jaw 1102, the operation member 1540 can close the second jaw 1102 at a large angle, and then forcefully close the second jaw 1102 to reliably clamp the body tissue even when the operation member 1540 is moved only a relatively small distance in the initial stage.

After the clamping operation, the jaw-inclined surface 11022 may have a gentle inclination angle at the distal end. That is, after the operation member 1540 closes the second jaw 1102, the operation member 1540 may move linearly along the second jaw 1102 without applying force to the second jaw 1102.

### Before Clamping Operation Performed by Operation Member

FIG. 31 is a view illustrating a state before the operation member 1540 of FIG. 23 performs an operation (clamping operation) of closing the jaws 1103, and FIG. 32 is an enlarged view of portion Z1 of FIG. 31.

Referring to FIGS. 31 and 32, the operation member 1540 may be disposed adjacent to the portion of the jaw 1103, at which the first jaw 1101 and the second jaw 1102 are axially coupled to each other, before the operation of closing the jaws 1103 is performed. In other words, the operation member 1540 may be disposed in a neutral position (the position before moving) before the operation of closing the jaws 1103 is performed. At this time, the first flange 1542 of the operation member 1540 may be or not be in contact with the jaw-inclined surface 11022 of the second jaw 1102. In other words, since the operation member 1540 has not yet begun to move, the second jaw 1102 remains in a state in which no force is applied thereto regardless of whether the operation member 1540 is in contact with the jaw-inclined surface 11022.

### During Clamping Operation Performed by Operation Member

FIG. 33 is a view illustrating a state in which the operation member 1540 of FIG. 23 performs the operation (clamping operation) of closing the jaws 1103, and FIG. 34 is an enlarged view of portion Z2 of FIG. 33.

Referring to FIGS. 33 and 34, the operation member 1540 may perform the operation of closing the second jaw 1102 while moving in the direction toward the distal end of the jaw 1103.

As the operation member 1540 is moved in the direction toward the distal end of the jaw 1103, the first flange 1542 of the operation member 1540 may come into contact with the jaw-inclined surface 11022. That is, as the operation member 1540 is moved in the direction toward the distal end of the jaw 1103, the first flange 1542 may move along the jaw-inclined surface 11022 while coming into contact with the jaw-inclined surface 11022. In other words, as the operation member 1540 is moved forward, the first flange 1542 is also moved forward, causing the second jaw 1102 to be moved downward by the first flange 1542. In other words, it may be described that as the operation member 1540 is moved forward, the first flange 1542 rides on and slides upward along the jaw-inclined surface 11022 of the second jaw 1102 while remaining in contact therewith.

At this time, the first flange 1542 may include the first flange curved part 15421, and in this case, the first flange 1542 may easily slide upward while remaining in contact with the jaw-inclined surface 11022. Accordingly, excessive friction may be prevented between the first flange 1542 and the jaw-inclined surface 11022, allowing the first flange 1542 to slide smoothly upward along the jaw-inclined surface 11022.

As the first flange 1542 of the operation member 1540 is moved while coming into contact with the jaw-inclined surface 11022 of the second jaw 1102, the operation member 1540 may apply a force to the second jaw 1102. The second jaw 1102 may receive the force in the direction of rotation by the first flange 1542, which may cause the second jaw 1102 to close. That is, the second jaw 1102 may rotate in a direction to move relatively closer to the first jaw 1101.

As described above, the jaw-inclined surface 11022 of the second jaw 1102 may have a steeply upwardly inclined portion disposed at the proximal end thereof. Accordingly, the first flange 1542 may first come into contact with the steeply upwardly inclined portion of the jaw-inclined surface 11022. In other words, when the operation member 1540 begins to move toward the distal end of the jaw 1103, the first flange 1542 may come into contact with the jaw-inclined surface 11022, forming a large inclination angle therewith. This means that even when the operation member 1540 is moved a small distance, the operation member 1540 can close the second jaw 1102 to the same extent as closing the second jaw 1102 with a larger angle.

Thereafter, as the operation member 1540 is moved along the distal end of the jaw 1103, the first flange 1542 may form a relatively small inclination angle with the jaw-inclined surface 11022. For example, the first flange 1542 may apply a force to the jaw-inclined surface 11022 while forming an angle of 5° to 15° with the jaw-inclined surface 11022.

Since the jaw-inclined surface 11022 is formed gently at the distal end of the jaw-inclined surface 11022, when the first flange 1542 reaches the distal end, the first flange 1542 applies a relatively large force to the jaw-inclined surface 11022. Here, the direction of the force that the first flange 1542 applies to the jaw-inclined surface 11022 refers to the direction in which the first flange 1542 applies a force to the jaw-inclined surface 11022 to cause the second jaw 1102 to close.

Accordingly, when the first flange 1542 reaches the distal end of the jaw-inclined surface 11022, the second jaw 1102 approaches the closed state, which allows the first flange 1542 to continue moving toward the distal end of the jaw-inclined surface 11022 while applying a relatively greater force to clamp the second jaw 1102 in the direction of closing the jaws 1103 compared to when the first flange 1542 is at the proximal end of the jaw-inclined surface 11022.

### After Clamping Operation Performed by Operation Member

FIG. 35 is a view illustrating a state after the operation member 1540 of FIG. 23 performs the operation (clamping operation) of closing the jaws 1103, and FIG. 36 is an enlarged view of portion Z3 of FIG. 35.

Referring to FIGS. 35 and 36, the operation member 1540 may move toward the distal end of the jaw 1103 after performing the operation of closing the jaws 1103. In other words, after the jaws 1103 are completely closed, the operation member 1540 may move toward the distal end of the jaw 1103.

Since the first flange 1542 has passed the jaw-inclined surface 11022 of the second jaw 1102, the first flange 1542 of the operation member 1540 may come into contact with the upper surface of the second jaw 1102 and slidably move toward the distal end of the second jaw 1102. That is, the operation member 1540 may perform an operation such as stapling while moving toward the distal end of the jaw 1103.

### (Correlation Between Operation of Operation Member and Force Applied to Operation Member

Hereinafter, a specific embodiment of the operation member 1540 will be described in detail.

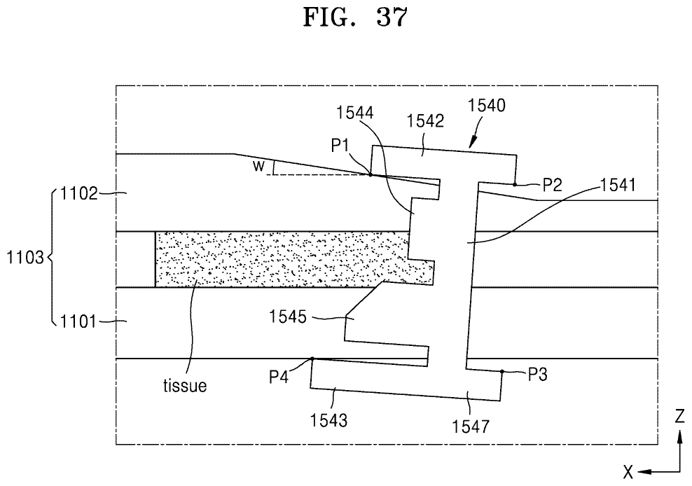

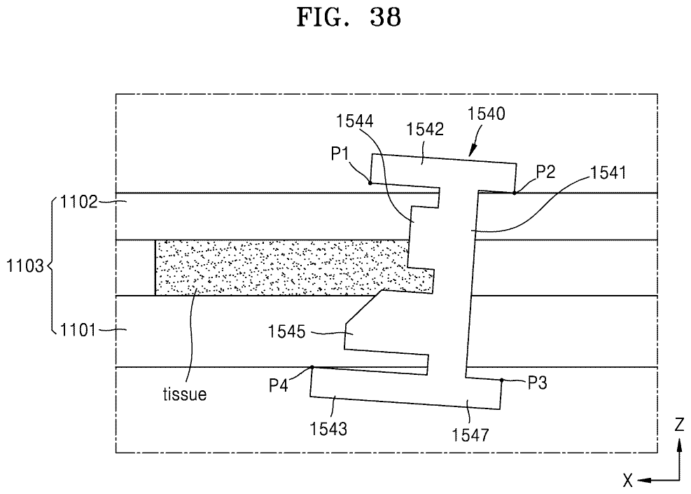

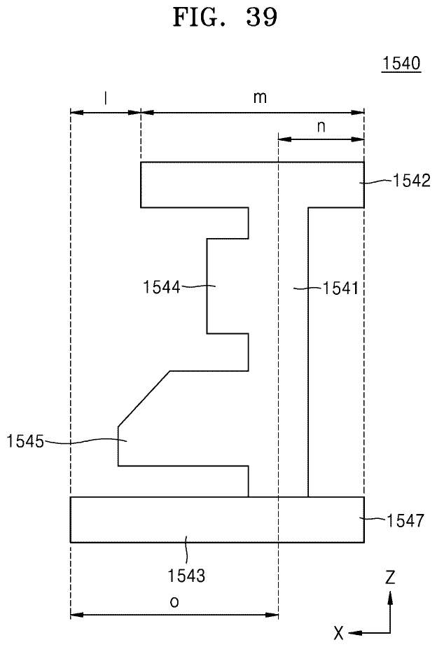

FIG. 37 is a view for describing operations of the operation member 1540 when the operation member 1540 performs the operation (clamping operation) of closing the jaws 1103, FIG. 38 is a view for describing operations of the operation member 1540 when the operation member 1540 performs an operation of moving along the jaw 1103, and FIG. 39 is a side view schematically illustrating the operation member 1540 of FIGS. 37 and 38.

Referring to FIGS. 37 to 39, the operation member 1540 may include the body 1541 and at least one flange.

The operation member 1540 may include the first flange 1542 disposed on one side of the body 1541, the second flange 1543 disposed on another side of the body 1541, and the third flange 1547 disposed in a direction parallel to the second flange 1543. In addition, the operation member 1540 may further include the wedge 1545 and the blade 1544.

Since the contents of the body 1541, the first flange 1542, the second flange 1543, the third flange 1547, the wedge 1545, and the blade 1544 are the same as those described above, and thus will not be described in detail herein.

As described above, the width of the body 1541 may be formed to be less than the width of each of the first slit 11011 and the second slit 11021. Thus, the occurrence of unnecessary friction, which may be generated when the body 1541 and the first and second slits 11011 and 11011 come into close contact or contact each other as the operation member 1540 is moved in the longitudinal direction of the jaw 1103, may be prevented. In other words, it may be described that, when the operation member 1540 is moved in the longitudinal direction of the jaw 1103, the body 1541 does not receive a repulsive force caused by friction from the first slit 11011 and the second slit 11021.

Meanwhile, during surgery, the patient's body tissue may apply a repulsive force to the operation member 1540. For example, as the blade 1544 advances through the body tissue while incising the body tissue, the operation member 1540 may receive a repulsive force from the body tissue. In addition, the operation member 1540 may receive a repulsive force when at least a portion thereof comes into contact with the body tissue, even after the blade 1544 has incised the body tissue.

Thus, it should first be noted that, for convenience of description, FIGS. 37 to 39 are conceptual views mainly illustrating the portion where the operation member 1540 interacts with the jaw 1103 or the patient's body tissue in relation to force. In other words, it is of course possible that the body 1541 may have a larger or smaller width in the X-axis direction compared to that illustrated in FIGS. 37 to 39.

The operations of the operation member 1540 when the operation member 1540 performs the operation of closing the jaws 1103 will be described with reference to FIG. 37.

Referring to FIG. 37, when performing the operation of closing the jaws 1103, the operation member 1540 may be subjected to a rotational force in a direction that causes the second flange 1543 to move forward further toward the distal end than the first flange 1542. For example, it may be described that the operation member 1540 is subjected to a rotational force in the clockwise direction based on FIG. 37.

When the operation member 1540 performs the operation of closing the jaws 1103, a distal end P1 of the first flange 1542 of the operation member 1540 may come into contact with the jaw-inclined surface 11022 of the second jaw 1102. In addition, a proximal end P2 of the first flange 1542 of the operation member 1540 may not be in contact with the jaw-inclined surface 11022 of the second jaw 1102. In addition, a proximal end P3 of the third flange 1547 of the operation member 1540 may not be in contact with the first jaw 1101. In addition, a distal end P4 of the second flange 1543 of the operation member 1540 may come into contact with the first jaw 1101. Accordingly, the operation member 1540 may press the second jaw 1102 in a direction toward the first jaw 1101, and the second jaw 1102 may be closed.

Forces applied to the operation member 1540 when the operation member 1540 performs the operation of closing the jaws 1103 will be discussed in more detail.

First, a driving force may be applied to the operation member 1540 to move the operation member 1540 in the direction toward the distal end of the jaw 1103. In addition, due to the contact between the first flange 1542 and the jaw-inclined surface 11022 of the second jaw 1102, repulsive and frictional forces of the jaw-inclined surface 11022 may be applied to the operation member 1540. In addition, due to the contact between the second flange 1543 and the first jaw 1101, repulsive and frictional forces of the first jaw 1101 may be applied to the operation member 1540.

The magnitude of each of the forces applied to the operation member 1540 and the positions of the points at which these forces act on the operation member 1540 may all vary. This means that the forces applied to the operation member 1540 may cancel each other out, and ultimately, the sum of rotational moments applied to the operation member 1540 may become zero. In other words, when a force is applied to the operation member 1540 to move the operation member 1540, a rotational force may be applied to the operation member 1540. This rotational force may be generated by the repulsive forces between the jaws 1103 and the flanges and the frictional force acting on the operation member 1540. As such a rotational force becomes stronger, a force applied by the user to move the operation member 1540 is used to offset the rotational force. Thus, the operation member 1540 may encounter challenges in efficiently applying a force to the jaw 1103 and locked to the jaw 1103, and thus the operation member 1540 may become immobile, or excessive force may be required to move the operation member 1540. Accordingly, in order to efficiently close the jaws 1103 using the operation member 1540 in a clamping period of the jaw 1103, it is necessary to offset the rotational force applied to the operation member 1540 with a minimum force. For example, in order to efficiently close the jaws 1103 using the operation member 1540 in the clamping period of the jaw 1103, it is important to minimize the frictional force that is applied to the operation member 1540 caused by the rotational force applied to the operation member 1540.

The operations of the operation member 1540 when the operation member 1540 performs the operation of moving along the jaws 1103 will be described with reference to FIG. 38. That is, the operations of the operation member 1540 when the operation member 1540 is moved along the jaw 1103 after completely closing the jaws 1103 will be described.

Referring to FIG. 38, when moving in the direction toward the distal end of the jaw 1103 in a state in which the jaws 1103 are closed, the operation member 1540 may be subjected to a rotational force in a direction that causes the second flange 1543 to move further toward the distal end of the jaw 1103 than the first flange 1542. For example, it may be described that the operation member 1540 is subjected to a rotational force in the clockwise direction based on FIG. 38.

When the operation member 1540 is moved in the direction toward the distal end of the jaw 1103 in a state in which the jaws 1103 are closed, the distal end P1 of the first flange 1542 of the operation member 1540 may not be in contact with the second jaw 1102. In addition, the proximal end P2 of the first flange 1542 of the operation member 1540 may come into contact with the second jaw 1102. In addition, the proximal end P3 of the third flange 1547 of the operation member 1540 may not be in contact with the first jaw 1101. In addition, the distal end P4 of the second flange 1543 of the operation member 1540 may come into contact with the first jaw 1101. Accordingly, the operation member 1540 may move efficiently in the longitudinal direction of the jaw 1103.

Forces applied to the operation member 1540 when the operation member 1540 is moved in the longitudinal direction of the jaw 1103 in a state in which the jaws 1103 are closed will be described in detail.

First, a driving force may be applied to the operation member 1540 to move the operation member 1540 in the direction toward the distal end of the jaw 1103. In addition, due to the contact between the first flange 1542 and the second jaw 1102, repulsive and frictional forces of the second jaw 1102 may be applied to the operation member 1540. In addition, due to the contact between the second flange 1543 and the first jaw 1101, repulsive and frictional forces of the first jaw 1101 may be applied to the operation member 1540. In addition, repulsive and frictional forces caused by the patient's body tissue caught between the first jaw 1101 and the second jaw 1102 may be applied to the operation member 1540. In addition, a repulsive force of the blade 1544, which is generated as the blade 1544 cuts through the patient's body tissue, may be applied to the operation member 1540. In addition, a repulsive force generated when the wedge 1545 pushes the staple may be applied to the operation member 1540.

The magnitude of each of the forces applied to the operation member 1540 and the positions of the points at which these forces act on the operation member 1540 may all vary. This means that some of the forces applied to the operation member 1540 may cancel each other out, and ultimately, a rotational moment may be applied to the operation member 1540. In other words, when a force is applied to move the operation member 1540, a rotational moment may act on the operation member 1540, which may result in a rotational force. As the rotational force becomes stronger, the operation member 1540 may encounter challenges in efficiently applying a force to the jaw 1103 and locked to the jaw 1103, and thus the operation member 1540 may become immobile, or excessive force may be required to move the operation member 1540. Thus, it may be necessary to offset the rotational force applied to the operation member 1540 with a minimum force in order to efficiently move the operation member 1540 in a moving section of the jaw 1103. For example, in order to efficiently move the operation member 1540 in the moving section of the jaw 1103, it may be important to minimize the frictional force applied to the operation member 1540 caused by the rotational force applied to the operation member 1540.

### Specific Embodiment of Operation Member

In the following description, a specific embodiment for offsetting the rotational force of the above-described operation member 1540 is presented.

The first flange 1542 may be formed to extend to a length of 2 mm to 10 mm. The end tool 1100 of the surgical instrument 1000 according to the present disclosure may be inserted into the patient's body to perform various motions. Thus, the first flange 1542 may be formed to a length of 2 mm to 10 mm to enable the end tool 1100 to perform a number of motions while inserted inside the patient's body.

In an embodiment, the operation member 1540 may be formed such that the first flange 1542 extends further in the direction toward the distal end than the first flange 1542. In other words, the second flange 1543 may be formed to protrude further in the direction toward the distal end of the jaw 1103 (in the X-axis direction) than the first flange 1542 based on FIG. 39. In other words, it may be described that, when a length of the first flange 1542 is referred to as m [mm] (hereinafter referred to as a length m), and a length by which the second flange 1543 protrudes further than the distal end of the first flange 1542 is referred to as l [mm] (hereinafter referred to as a length l), the length l [mm] may have a positive value.

As a specific embodiment, in the operation member 1540, the second flange 1543 may extend further than the first flange 1542 such that the second flange 1543 has a length value that causes the sum of the rotational moments applied to the operation member 1540 to be zero. Here, of directions of the rotational moment, a positive direction refers to a direction of the rotational moment that attempts to rotate the operation member 1540 in the clockwise direction based on FIG. 37, and a negative direction refers to a direction of the rotational moment that attempts to rotate the operation member 1540 in the counterclockwise direction based on FIG. 37. That is, the rotational moment applied to the operation member 1540 is determined by the length l [mm]. At this time, when the value of l [mm] at which the sum of the rotational moments applied to the operation member 1540 becomes zero is referred to as Deq [mm], the second flange 1543 may extend such that the length l [mm] has a value greater than Deq [mm].

Accordingly, when the operation member 1540 performs the operation of closing the jaws 1103, friction applied to the operation member 1540 may be minimized.

Describing the above in detail, as the operation member 1540 is moved, a rotational force is generated in the operation member 1540. In this case, the rotational force applied to the operation member 1540 is offset by an external force, so that the operation member 1540 may be moved toward the distal end of the jaw 1103 without rotating. For example, in a state in which the operation member 1540 and the jaws 1103 are at least partially in contact with each other, the operation member 1540 and the jaws 1103 apply force toward each other. and this force acts as an external force that offsets the rotational force applied to the operation member 1540. At this time, the stronger the rotational force applied to the operation member 1540, the stronger the external force to offset the rotational force, which causes a large amount of friction between the operation member 1540 and the jaw 1103. Accordingly, the operation member 1540 according to the present embodiment is formed in shape to receive a minimum rotational force, thereby ultimately minimizing the frictional force applied to the operation member 1540.

Specifically, the operation member 1540 may naturally be subjected to a rotational force in the clockwise direction based on FIG. 37 while moving toward the distal end of the jaw 1103. At this time, as the value of l [mm] of the operation member 1540 increases, a rotational moment additionally applied to the operation member 1540 may have a negative value, and accordingly, a positive rotational moment applied to the operation member 1540 may be offset. Meanwhile, when the operation member 1540 is rotated in the counterclockwise direction when the operation member 1540 is moved toward the distal end of the jaw 1103, the proximal end of the third flange 1547 of the operation member 1540 (in the embodiment described with reference to FIGS. 29 and 30, the proximal end of the second flange 1543′) comes into contact with the first jaw 1101, and thus, when the total rotational moment applied to the operation member 1540 has a negative value, a contact surface of the operation member 1540, which comes into contact with the jaw 1103, becomes a portion on which an external force acts to offset the rotational force applied to the operation member 1540, while also functioning as a friction surface. Accordingly, in the operation member 1540 according to the present embodiment, the second flange 1543 is formed such that the value of l [mm] is greater than Deq [mm], so that a negative rotational moment may act on the operation member 1540, and accordingly, the contact surface of the operation member 1540, which comes into contact with the jaw 1103, may become a portion of the operation member 1540 to which an external force applied, so that an unnecessary friction surface can be avoided, and the frictional force can be minimized.

In an embodiment, the length m [mm] of the first flange 1542 and the length l [mm] by which the second flange 1543 extends further than the first flange 1542 may satisfy a relationship of m/l<10. This may be seen as reflecting the possibility of variation in the length m [mm] of the first flange 1542. In other words, the length m [mm] of the first flange 1542 may be formed in various lengths according to the standard of the end tool 1100 of the surgical instrument 1000.

Meanwhile, as the sum of the length l [mm] by which the second flange 1543 extends further than the first flange 1542 and the length m [mm] of the first flange 1542 increases, the friction applied to the operation member 1540 becomes smaller.

However, since the end tool 1100 of the surgical instrument 1000 according to the present disclosure performs various tasks in the human body, the values of m [mm] and l [mm] are limited in range due to characteristics thereof.

Thus, as described above, by applying a negative rotational moment to the operation member 1540, the length m [mm] of the first flange 1542 and the length l [mm] by which the second flange 1543 extends further than the first flange 1542 may satisfy the relationship of m/l<10, and preferably a relationship of m/l <3, to allow the end tool 1100 to operate smoothly within the human body while generating minimal friction.

In an embodiment, when an inclination angle of the jaw-inclined surface 11022 when the first flange 1542 comes into contact with the jaw-inclined surface 11022 is referred to as w [degree], the inclination angle w [degree] of the jaw-inclined surface 11022 and the length l [mm] by which the second flange 1543 extends further than the first flange 1542 may satisfy a relationship of w/1<5 [degree/mm]. This may be seen as reflecting the possibility of variation in the inclination angle of the jaw-inclined surface 11022.

In other words, as described above, the jaw-inclined surface 11022 may be formed such that the inclination angle w [degree] varies in the longitudinal direction of the jaw 1103 in order for the operation member 1540 to efficiently close the second jaw 1102. Accordingly, when the inclination angle w [degree] of the jaw-inclined surface 11022 varies as the operation member 1540 is moved along the jaw-inclined surface 11022, the value of the rotational moment applied to the operation member 1540 in a section where the operation member 1540 closes the jaw 1103 may vary. In this case, the value of the required length by which the second flange 1543 extends further than the first flange 1542 may also vary in order to ensure that minimal friction is applied to the operation member 1540 while always applying a rotational moment to the operation member 1540 in the counterclockwise direction even though the inclination angle w [degree] of the portion of the jaw-inclined surface 11022, which come into contact with the first flange 1542, is changed. Accordingly, by forming the jaw-inclined surface 11022 and the second flange 1543 such that the inclination angle w [degree] of the jaw-inclined surface 11022 and the length l [mm] by which the second flange 1543 extends further than the first flange 1542 satisfy the relationship w/l<5 [degree/mm], while the operation member 1540 is moved along the jaw-inclined surface 11022, minimal friction may be applied to the operation member 1540, thereby allowing a clamping motion of the jaw 1103 inside the patient's body to be performed efficiently.

In an embodiment, when a horizontal distance from a rotation axis of the operation member 1540 to the proximal end of the first flange 1542 is referred to as n [mm], and a horizontal distance from the rotation axis of the operation member 1540 to the distal end of the second flange 1543 is referred to as o [mm], a relationship of n+o>10 [mm] may be satisfied.

Here, the rotation axis may refer to the center of rotation (or rotation attempted by the rotational force) of the operation member 1540 when the operation member 1540 is rotated (or is subjected to a rotational force) in the clockwise or counterclockwise direction based on FIG. 28 or the like. Here, the rotation axis may vary in position depending on the length of each of the flanges 1542 and 1543 forming the operation member 1540, the height of the operation member 1540, and the like.

When the operation member 1540 is moved after the jaw 1103 is clamped, a greater variety of forces may be applied to the operation member 1540 compared to the clamping motion of the jaw 1103. For example, when the operation member 1540 is moved in the direction toward the distal end of the jaw 1103, a variety of forces may act on the operation member 1540, including a force to move the operation member 1540 forward, frictional and repulsive forces received from the jaw 1103, a repulsive force received from the blade 1544, a repulsive force received from the wedge 1545, and the like. As such, as the above-described forces are applied, a rotational force is still applied to the operation member 1540. It may be said that it is ultimately desirable for the operation member 1540 to move linearly in the longitudinal direction of the jaw 1103 while efficiently offsetting the applied rotational forces, even after the jaws 1103 are clamped.

To this end, the operation member 1540 may satisfy the relationship n+o>10 [mm] to minimize the driving force for moving the operation member 1540.

In other words, while moving in the longitudinal direction of the jaw 1103, the operation member 1540 receives repulsive and frictional forces from the first jaw 1101, the second jaw 1102, the patient's body tissues, and the like, and thus is subjected to a rotational force. Thus, as the rotational force becomes stronger, excessive rotational force may be applied to the operation member 1540, and the contact between the operation member 1540 and the first and second jaws 1101 and 1102 becomes stronger, which increases the frictional force, and excessive force is required to offset the rotational force applied to the operation member 1540. Increasing the rotational force applied to the operation member 1540 ultimately results in a higher driving force required to move the operation member 1540. Thus, in order to effectively offset the rotational force, the sum of the horizontal distance n [mm] from the rotation axis of the operation member 1540 to the proximal end of the first flange 1542 and the horizontal distance o [mm] from the rotation axis of the operation member 1540 to the distal end of the second flange 1543 may be greater than 10 mm. In addition, by satisfying the relationship n+o>10 [mm], the end tool 1100 of the surgical instrument 1000 according to the present disclosure can be manufactured to a size that may perform various motions inside the patient's body.

As described above, the present disclosure has been described with reference to the embodiment described with reference to the drawings, but it will be understood that this is merely exemplary, and those of ordinary skill in the art will understand that various modifications and other equivalent embodiments are possible therefrom. Accordingly, the true technical protection scope of the present disclosure should be defined by the technical spirit of the appended claims.

The particular implementations shown and described herein are illustrative examples of the embodiments and are not intended to otherwise limit the scope of the embodiments in any way. For the sake of brevity, conventional electronics, control systems, software development and other functional aspects of the systems may not be described in detail. Further, the connecting lines or connectors shown in the drawings are intended to represent example functional relationships and/or physical or logical couplings between the various elements. It should be noted that many alternative or additional functional relationships, physical connections, or logical connections may be present in a practical device. In addition, no item or component is essential to the practice of the present disclosure unless the component is specifically described as “essential” or “critical”.

The use of the terms “a” and “an” and “the” and similar referents in the context of describing the present disclosure (especially in the context of the following claims) are to be construed to cover both the singular and the plural. Further, recitation of ranges of values herein are merely intended to serve as a shorthand method of referring individually to each separate value falling within the range, unless otherwise indicated herein, and each separate value is incorporated into the specification as if it were individually recited herein. Finally, operations of all methods described herein can be performed in any suitable order unless otherwise indicated herein or otherwise clearly contradicted by context. The present disclosure is not necessarily limited to the described order of the operations. The use of any and all examples, or exemplary terms (e.g., “such as”) provided herein, is intended merely to better illuminate the present disclosure and does not pose a limitation on the scope of the present disclosure unless otherwise claimed. Further, numerous modifications and adaptations will be readily apparent to one of ordinary skill in the art without departing from the spirit and scope of the present disclosure.

The present disclosure can provide a surgical instrument capable of efficiently moving an operation member along a jaw by applying a minimum load to the operation member.

However, the effect is illustrative and the effect of the present disclosure is not limited thereto.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. A surgical instrument comprising an end tool that rotates in at least one direction, wherein the end tool includes:
a pair of jaws including a first jaw and a second jaw, which are rotatable with respect to each other, wherein the first jaw includes a first slit formed in a longitudinal direction, and the second jaw includes a second slit formed in the longitudinal direction; and
an operation member including a body accommodated in the first slit and the second slit, a first flange formed on one side of the body, and a second flange formed on another side of the body, and moving in the longitudinal direction of the jaw,
wherein the operation member performs an operation of closing the pair of jaws while moving from a proximal end of the pair of jaws toward a distal end and an operation of linearly moving in the longitudinal direction of the pair of jaws while the pair of jaws are closed.

2. The surgical instrument of claim 1, wherein the second jaw includes a jaw-inclined surface formed to be inclined upward in a direction toward the distal end of the second jaw.

3. The surgical instrument of claim 2, wherein the jaw-inclined surface is formed such that an inclination angle changes in the longitudinal direction.

4. The surgical instrument of claim 3, wherein the jaw-inclined surface is formed such that an inclination angle at a proximal end is greater than an inclination angle at a distal end.

5. The surgical instrument of claim 2, wherein:
a distance between a surface of the first flange that comes into contact with the second jaw and a surface of the second flange that comes into contact with the first jaw is formed to have a value greater than or equal to that of a vertical distance between the first jaw and the second jaw; and
the operation member is subjected to a rotational force while moving in the longitudinal direction of the pair of jaws.

6. The surgical instrument of claim 5, wherein, when performing the operation of closing the pair of jaws, the operation member is subjected to a rotational force in a direction that causes the second flange to move forward further toward the distal end of the pair of jaws than the first flange.

7. The surgical instrument of claim 6, wherein, when the operation member performs the operation of closing the pair of jaws, a distal end of the first flange comes into contact with the jaw-inclined surface, and a distal end of the second flange comes into contact with the first jaw.

8. The surgical instrument of claim 5, wherein, when the operation member is moved in a direction toward the distal end of the pair of jaws while the pair of jaws are closed, the second flange is subjected to a rotational force in a direction that causes the second flange to move forward further toward the distal end than the first flange.

9. The surgical instrument of claim 8, wherein, when the operation member is moved in the direction toward the distal end of the pair of jaws while the pair of jaws are closed, a proximal end of the first flange comes into contact with the second jaw and a distal end of the second flange comes into contact with the first jaw.

10. The surgical instrument of claim 5, wherein a relationship of n+o>10 mm is satisfied where n [mm] is a horizontal distance from a rotation axis of the operation member to a proximal end of the first flange, and o [mm] is a horizontal distance from the rotation axis of the operation member to a distal end of the second flange.

11. The surgical instrument of claim 2, wherein the operation member is formed such that the second flange extends further in a direction toward the distal end than the first flange.

12. The surgical instrument of claim 11, wherein the first flange has a length of about 2 mm to about 10 mm.

13. The surgical instrument of claim 11, wherein a relationship of m/l<10 is satisfied where m [mm] is a length of the first flange and l [mm] is a length by which the second flange extends further than the first flange.

14. The surgical instrument of claim 11, wherein the second flange extends further than the first flange to a length whose value is greater than a value that causes a sum of rotational moments applied to the operation member to be zero.

15. The surgical instrument of claim 11, wherein a relationship of w/l<5 [degree/mm] is satisfied where w [degree] is an inclination angle of the jaw-inclined surface and l [mm] is a length by which the second flange extends further than the first flange.
